(19) United States
(12) Patent Application Publication
(10) Pub. No.: US 2014/0356899 A1
(43) Pub. Date: Dec. 4, 2014

(54) Biological Contamination Modeling of Surfaces in Hot Humid Air

(71) Applicants: Tony L. Buhr, King George, VA (US); Claire M. Wells, Alexandria, VA (US); Alice A. Young, Fredericksburg, VA (US); Edward J. Prokop, III, King George, VA (US); John R. Crigler, Fredericksburg, VA (US)

(72) Inventors: Tony L. Buhr, King George, VA (US); Claire M. Wells, Alexandria, VA (US); Alice A. Young, Fredericksburg, VA (US); Edward J. Prokop, III, King George, VA (US); John R. Crigler, Fredericksburg, VA (US)

(73) Assignee: United States of America as represented by the Secretary of the Navy, Arlington, VA (US)

(21) Appl. No.: 14/243,789

(22) Filed: Apr. 2, 2014

Related U.S. Application Data
(60) Provisional application No. 61/807,896, filed on Apr. 3, 2013.

(51) Int. Cl. C12Q 1/22; G06F 19/24

(52) U.S. Cl. CPC C12Q 1/22; G06F 19/24
USPC 435/31; 702/19

(57) ABSTRACT

A protocol process is provided for testing and evaluating Bacillus spores. The process includes isolating a sample of the spores, providing an environmental matrix of exposure condition elements, determining a midpoint for each control range between the low and high extremities, selecting condition elements from the matrix, with each condition element corresponding to a first condition, subjecting the sample to the condition elements from the matrix, counting survival spores for each condition element, performing a response surface methodology (RSM) analysis from the survival spores, and determining coefficients that satisfy a relation for k conditions. The matrix has control ranges of temperature, relative humidity and exposure duration, the control ranges extending from a low extremity to a high extremity. The first condition is one of the low and high range extremities and remaining conditions occupying the midpoint for a corresponding control range. All condition elements are uniquely distinguishable. The relation can be expressed as:

$$ln ( \frac{p ( x )}{1 - p ( x )} ) = {β}_{0} + {\sum }_{j = 1}^{k} {β}_{j} {x}_{j} + {\sum }_{j = 1}^{k} {β}_{jj} {x}_{j}^{2} + {\sum }_{i < j}^{ } {\sum }_{j = 2}^{k} {β}_{ij} {x}_{i} {x}_{j} + b .$$

The proportion response p(x) of inactivated spores quantifies long odds spore inactivation, xⱼ represents a set of independent variables, βⱼ denotes a correlation coefficient from j=0, 1, . . . k, and b represents a correlation blocking term.

### CROSS REFERENCE TO RELATED APPLICATION

Pursuant to 35 U.S.C. §119, the benefit of priority from provisional application 61/807,896, with a filing date of Apr. 3, 2013, is claimed for this non-provisional application.

### STATEMENT OF GOVERNMENT INTEREST

The invention described was made in the performance of official duties by one or more employees of the Department of the Navy, and thus, the invention herein may be manufactured, used or licensed by or for the Government of the United States of America for governmental purposes without the payment of any royalties thereon or therefor.

### BACKGROUND

The invention relates generally to predictive modeling of biological decontamination effectiveness. In particular, the invention relates to quantitative performance prediction of decontaminating treatments of surfaces exposed to hot humid air.

Deliberate attacks using pathogens have initiated interest in predictive capability for decontamination effectiveness. Lessons learned following the 2001 anthrax attacks highlighted the need to improve biological decontamination. For example, the three chemical fumigants employed for decontamination (vaporous hydrogen peroxide, paraformaldehyde and chlorine dioxide) were effective sporicides, but damaged many materials (Holwitt et al. 2000; Anon 2003, 2004; Buhr et al. 2011, 2012). As a result, post-decontamination disposal of destroyed materials was complicated (Canter 2005). The cost of decontamination following the 2001 attacks exceeded 300 million US dollars, and the total remediation took 34 months (Buhr et al. 2012).

Thermal decontamination is a potential chemical-free method of remediation that may be less damaging to materials (Decker et al. 1954; Ehrlich et al. 1970; Holwitt et al. 2000). The combined effect of heat, humidity and pressure in an autoclave is an example of thermal decontamination. Higher temperatures, higher humidity and higher pressures enable reduced sterilization times.

Previous work examining thermal spore inactivation suggests that low water content (0.6 gram of water per gram of dry protein), dipicolinic acid content and small acid-soluble spore proteins contribute to high heat resistance of spores (Murrell and Scott 1966; Alderton and Snell 1970; Gerhardt and Marquis 1989; Melly et al. 2002; Coleman et al. 2007; Sunde et al. 2009; Zhang et al. 2010). Spores could have additional mechanisms to resist heat such as expulsion of water from the protoplast as observed in Bacillus stearothermophilus (Prokop and Humphrey 1972). Active water removal may prevent irreversible protein aggregation and shield dry spores from harm, while the hydration achieved with humid conditions may promote irreversible protein aggregation and lead to spore death (Sunde et al. 2009). In addition, protein denaturation of heatlabile metabolic enzymes is also a generally accepted effect of thermal decontamination (Coleman et al. 2007).

The goals here were to develop test methods and justify a simulant in order to characterize and quantify the efficacy of hot, humid air inactivation of Bacillus spores dried on six different materials. Prior to testing, a spore preparation protocol was developed, useful for both B. anthracis ΔSterne and B. thuringiensis Al Hakam, to eliminate spore preparation methodology as a source of experimental variability. Test methods were developed to control temperature and relative humidity while safely containing spores.

The experimental design was guided by response surface methodology (RSM) in order to keep the number of tests manageable while producing meaningful data and conclusions. RSM integrates statistical design of experiments (DOE) fundamentals, regression modeling techniques and optimization methods. RSM is an iterative method widely used in industry where the aim is to use a sequence of designed experiments to obtain an optimal response (Beauregard et al. 1992; Montgomery 2009; Myers et al. 2009).

### SUMMARY

Hot, humid air decontamination is being explored at the limits of sporicidal efficacy as a decontamination technology with improved materials compatibility. Various exemplary embodiments focus on describing the application of RSM to experimental design and data analysis of the hot, humid air test data. The objective through this disclosure involves mathematical description of the effectiveness of hot, humid air decontamination, thus enabling an end-user to find acceptable operating conditions depending on the material to be decontaminated.

Conventional test and analysis protocols yield disadvantages addressed by various exemplary embodiments of the present invention. In particular, various exemplary embodiments provide a protocol for testing and analyzing comparative survival of Bacillus spores. The protocol includes isolating a sample of the spores, providing an environmental matrix of exposure condition elements, determining a midpoint for each control range between the low and high extremities, selecting condition elements from the matrix, with each condition element corresponding to a first condition, subjecting the sample to the condition elements from the matrix, counting survival spores for each condition element, performing a response surface methodology (RSM) analysis from the survival spores, and determining coefficients that satisfy a relation for k conditions.

The matrix has control ranges of temperature, relative humidity and exposure duration, the control ranges extending from a low extremity to a high extremity. The first condition is one of the low and high range extremities and remaining conditions occupying the midpoint for a corresponding control range. All condition elements are uniquely distinguishable. The relation can be expressed as:

$$ln ( \frac{p ( x )}{1 - p ( x )} ) = {β}_{0} + {\sum }_{j = 1}^{k} {β}_{j} {x}_{j} + {\sum }_{j = 1}^{k} {β}_{jj} {x}_{j}^{2} + {\sum }_{i < j}^{ } {\sum }_{j = 2}^{k} {β}_{ij} {x}_{i} {x}_{j} + b .$$

The proportion response p(x) of inactivated spores quantifies long odds spore inactivation, xⱼ represents a set of independent variables, βⱼ denotes a correlation coefficient from j=0, 1, . . . k, and b represents a correlation blocking term.

### BRIEF DESCRIPTION OF THE DRAWINGS

These and various other features and aspects of various exemplary embodiments will be readily understood with reference to the following detailed description taken in conjunction with the accompanying drawings, in which like or similar numbers are used throughout, and in which:

FIG. 1 is a schematic view of an equipment configuration diagram;

FIG. 2 is an isometric view of a response surface method parameter box;

FIG. 3 is a schematic view of a spore separation process diagram;

FIG. 4 is a tabular view of number of spore replicates;

FIG. 5 is a tabular view of quantities of heat resistant spores;

FIG. 6 is a tabular view of spore size by equivalent diameters;

FIG. 7 is a plot view of spore particle size distribution;

FIGS. 8A and 8B are tabular views of cell divisions;

FIG. 9 is a tabular view of spore extraction efficiency;

FIGS. 10A and 10B are tabular views of log survival of spores;

FIG. 11 is a tabular view of test mean comparisons of spore survivals;

FIGS. 12A through 12L constitute a set of micrograph views of spores;

FIGS. 13A through 13J are perspective response surface views of spore survivals;

FIGS. 14A through 14E are perspective views of inactivation diagrams;

FIGS. 15A through 151 are contour views of spore survival responses;

FIGS. 16A through 16U are tabular views of parameters and coefficients;

FIG. 17 is a tabular view of coefficients for the correlation relation;

FIGS. 18A through 18H are contour plot views of B. anthracis on APC;

FIGS. 19A through 19H are contour plot views of B. thuringinesis on APC;

FIGS. 20A through 20H are contour plot views of B. anthracis on nylon;

FIGS. 21A and 21B are contour plot views of B. thuringinesis on nylon;

FIGS. 22A and 22B are coltour profile plots of B. anthracis and B. thuringinesis;

FIG. 23 is a tabular view of blocking variable values; and

FIG. 24 is a tabular view of coefficients for the equation relations.

### DETAILED DESCRIPTION

In the following detailed description of exemplary embodiments of the invention, reference is made to the accompanying drawings that form a part hereof, and in which is shown by way of illustration specific exemplary embodiments in which the invention may be practiced. These embodiments are described in sufficient detail to enable those skilled in the art to practice the invention. Other embodiments may be utilized, and logical, mechanical, and other changes may be made without departing from the spirit or scope of the present invention. The following detailed description is, therefore, not to be taken in a limiting sense, and the scope of the present invention is defined only by the appended claims.

There is a need, therefore, to develop effective decontaminants for biological agents with improved materials compatibility. To this end, test methods were developed to show that hot, humid air effectively inactivates B. anthracis ΔSterne and B. thuringiensis Al Hakam spores with similar kinetics. Spores (>7 logs) of both strains were dried on six different test materials. In particular, response surface methodology (RSM) was employed to identify the limits of spore survival at optimal test combinations of temperature (60° C., 68° C., 77° C.), relative humidity (60% H_{R}, 75% H_{R}, 90% H_{R}) and time (1 day, 4 days, 7 days). No spores survived the harshest test run (77° C., 90% H_{R}, 7 days), while >6.5 logs of spores survived the mildest test run (60° C., 60% H_{R}, 1 day). Spores of both strains inoculated on nylon webbing and polypropylene had greater survival rates at 68° C., 75% H_{R}, 4 days than spores on other materials. Electron microscopy showed no obvious physical damage to spores using hot, humid air, which contrasted with pH-adjusted bleach decontamination. Hot, humid air is a potential alternative to conventional chemical decontamination.

In support of this effort, RSM has been employed using a face-centered cube design (FCD) to describe and predict spore inactivation of Bacillus anthracis ΔSterne and B. thuringiensis Al Hakam spores after exposure to hot, humid air. For each strain-substrate pair, an attempt was made to fit a first or second order model. All three independent predictor variables (temperature, relative humidity, and time) were significant in the models except for B. thuringiensis Al Hakam on nylon where time was not significant.

Modeling could not be accomplished for wiring insulation and wet spores, cases with complete spore inactivation in the majority of the experimental space. In cases where a predictive equation could be fit, example response surface plots with time set to four days were generated. The survival of highly purified Bacillus spores can be predicted for most materials tested if given the settings for temperature, relative humidity, and time. Dependence of spore survival and predictability of this survival on material substrate was demonstrated.

Decontamination of materials, rooms and equipment contaminated with biological agents. This is especially applicable to sensitive equipment and aircraft. There is a need to develop new mild decontaminants with improved materials compatibility compared to existing decontaminants. Conventional operational decontamination procedures can and do destroy many pieces of sensitive equipment, including aircraft. Replacement decontaminants should preferably have properties of materials compatibility, little or no toxicity, little or no reagents, little or no environmental concerns. To this effect, this disclosure provides convenient and simple to use mathematical models based on time, temperature, and relative humidity for evaluation of such decontaminants for various environments.

Samples of Bacillus anthracis ΔSterne were obtained from the Unified Culture Collection at United States Army Medical Research Institute of Infectious Diseases (USAMRIID) in Frederick, Md. The source was Unified Culture Collection Identifier BAC1056, lot number CA062700A. Samples of Bacillus thuringiensis Al Hakam (isolated from the Iraqi Al Hakam facility) were provided by Johnathan Kiel at Brooks Air Force Base, San Antonio, Tex.

Samples of Bacillus atrophaeus ATCC 9372 and B. cereus ATCC 4342 spores were prepared as described in literature (Buhr et al. 2008). Bacillus thuringiensis Al Hakam and B. anthracis ΔSterne sporulation medium was 0.8% nutrient broth (NB) amended with culture collection of yeasts (CCY) salts (Stewart et al. 1981; Atrih and Foster 2001; Buhr et al. 2008) at pH 7.0. Preaerated and preheated sporulation medium (333 ml medium in 1-liter baffled Corning flasks with filter-caps) was inoculated with 10⁵-10⁶ spores ml⁻¹ and incubated at 34° C. with shaking (300 rev min⁻¹) for 72±2 hrs in a New Brunswick Scientific shaker/incubator. Cultures were amended with Tween 80 (final concentration 3%) and incubated an additional 24±2 hrs, 34° C. at 300 rev min⁻¹ to disperse spores, specifically to address B. thuringiensis Al Hakam spore hydrophobicity. Spores were harvested and characterized via heat-resistant titres, microscopy and Coulter analysis (Buhr et al. 2008; McCartt et al. 2011; Buhr et al. 2011).

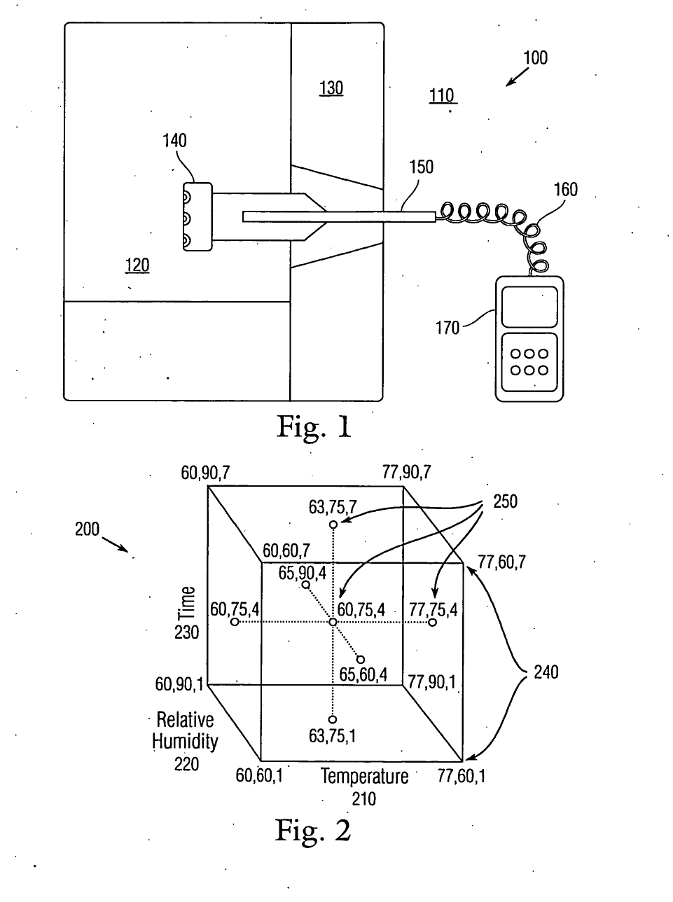

FIG. 1 illustrates a simplified diagram view 100 of an exemplary validation method. This process validates spore response in a consistent repeatable fashion for hot, humid air passes through 0.2-μm-filtered caps into the 50-ml conical tube 130 containing spore-inoculated test substrates. An external environment 110 surrounds an environmental chamber 120 having an inner wall 130. The chamber 120 includes a 0.2 μm filter-cap 140 held to the wall 130 by a Styrofoam plug 150. An electrically conductive wire 160 communicates signals from the filter-cap 140 to a hygrometer/thermometer 170. The interior dimensions of the exemplary chamber 120 used in the tests reported herein were 49 cm×38 cm×54 cm. The wall 130 was 14 cm in thickness. The conical tubes 130 were 3 cm diameter and 11.5 cm deep.

Set-up and validation of environmental test equipment included chambers for such purposes from Envirotronics in Grand Rapids, Mich., particularly LH0010 environmental test chambers to control temperature and relative humidity during testing. Fifty-milliliter (50 ml) polypropylene conical tubes capped with 0.2 μm filter-caps 140 from Techno Plastic Products in Trasadingen, Switzerland model TPP® TP87050 were selected for hot, humid air testing in order to enable air exchange while preventing spores from escaping the conical tube and to develop a test method that reduced handling of inoculated coupons during testing.

A traceable hygrometer/thermometer 170 from Control Company in Friendswood, Tex. was disposed within a single TPP® conical tube. The hygrometer was certified by National Institute of Standards and Technology (NIST). To construct the hygrometer—conical tube assembly, a hole was drilled through the conical tube bottom. The conical tube was inserted into a short piece of vinyl tubing (1.25-inch o.d.×1-inch i.d., annular dimensions defined by outer and inner diameters) and then secured using electrical tape and a 1.25-inch Mack washer from Parafilm® in Oshkosh, Wis. The hygrometer probe was then inserted into the conical tube such that the measurement point was the 37.5-ml gradation marking on the conical tube and sealed with Parafilm® and electrical tape. The assembly was fitted into a piece of 1.5-inch×2-inch polyvinylchloride and placed through the side port of the environmental chamber using a custom-cut Styrofoam plug 150. This assembly enabled real-time monitoring of temperature and relative humidity inside the conical tube exactly where test coupons would be located. After assembly, temperature and relative humidity were recorded every 5 min and compared to recordings from the built-in environmental chamber sensors. Recordings continued until the hygrometer measurements matched the chamber readings, or until they remained stable for at least 15 min.

Equipment included substrate coupons and sundry materials. Square 2×2 cm coupons of five different test substrates and the inside surface of 50-ml TPP® polypropylene conical tubes were used to contain the 2×2 cm test coupons inoculated with >7 logs of spores. These were aluminum 2024-T3 coupons from the Coatings Group at the University of Dayton Research Institute (UDRI), Daytob, Ohio. The coupons were painted with water-based aircraft performance coating (APC), and anti-skid material (black 60-grit anti-skid tape from No Skidding Products, Inc, Cheektowaga, N.Y. were applied.

Coupon insulation (DMS-2315-C, Type 2 Chase Facile Inc.) from InsulFab 315, Paterson, N.J. (InsulFab) was provided by Tim Provens of Wright-Patterson Air Force Base, Dayton, Ohio Wiring insulation in the form of Kapton Film Type HN, 1 mil, ref. no. 6197844-00 was purchased from Cole Parmer, Vernon Hills, Ill. Nylon webbing (nylon) was purchased from US Netting in Erie, Pa., and the ends of each coupon were cauterized to prevent fraying. Prior to testing, coupons were rinsed with 1 MΩ, de-ionized water, placed on absorbent paper in an autoclave-safe container and autoclaved on wet cycle for 30 min at 121° C. Autoclaved coupons were stored in sterile containers until used.

Response surface methodology (RSM): As in all design of experiment (DOE) arrangements, the experimental boundaries for RSM must be set by the researcher. Spore survival was the response to be modeled (i.e., the dependent variable). Time, temperature and relative humidity were the factors to be varied (i.e., the independent variables). Real-world limitations combined with RSM experimental design were used to select test combinations. The upper limit for temperature and relative humidity was initially set at 82° C., 90% relativity humidity, but was adjusted to 77° C., 90% relative humidity to comply with limits set by material manufacturers and because unpublished data already showed no spore survival at 82° C., 90% relative humidity for 6 hrs.

For the lower limits, the temperature was set at 60° C., 60% relative humidity where spores were known to survive for multiple days. In order to fit and analyze the response surface, three equally spaced values for each factor and a center point were tested as shown in FIG. 2 in view 200. Temperatures of 140° F., 155° F. and 170° F. were selected, converted to Celsius for reporting and then rounded to 60° C., 68° C. and 77° C. Each temperature—relative humidity combination was above the dew point to prevent condensation.

FIG. 2 shows a perspective view 200 of an exemplary design box for testing conditions. The RSM for experimental design involves three test factors: temperature ° C. on the horizontal axis 210, relative humidity % H_{R} on the into/out of axis 220, and exposure time in days) on the vertical axis 230. The design box is bounded by corner conditions 240 of environmental extremities. The center and face positions 250 provide exemplary conditions that isolate separate contributing environmental variables that may influence spore survival. The center point is 68° C., 75% H_{R}, 4 days.

Prior to coupon inoculation, concentrated spores were transferred from storage (−80° C.) into a water bath (50° C.) for at least 30 min to thoroughly thaw spores and maintain a consistent temperature for all inoculations. These spores were then vortexed for 15-30 sec and transferred to a 50-ml conical tube containing preheated (50° C.) aqueous 0.1% Tween 80. The volume of 0.1% Tween 80 was set to achieve a target concentration of 2×10⁸±1×10⁸ spores ml⁻¹. The diluted spore inoculum was held at 50° C. until coupon inoculation. At the time of inoculation, spores were vortexed again for 15-30 s and 0.1 ml of the spore inoculum was pipetted in a single drop directly onto each sterilized coupon. Inoculated coupons were left to dry overnight inside a biosafety cabinet.

Dried coupons were aseptically transferred to sterile 50-ml conical tubes equipped with TPP® 0.2 μm filter-caps 140 and stored at 22±3° C., ambient (40±20%) relative humidity prior to testing (room temperature, T_{R}). Polypropylene 50-ml conical tubes. equipped with TPP® 0.2 μm filter-caps 140 were also directly inoculated with 0.1 ml of spores and dried (polypropylene). These conical tubes served both as a test material and as a control to determine the impact of polypropylene on spore recovery.

Additionally, 0.1 ml of the spore inoculum was transferred to a solid-capped 50-ml polypropylene conical tube containing 4.9 ml of aqueous 0.1% Tween 80. These spores were kept wet and not dried (wet spores). Data from wet spores incubated at room temperature were compared with the original inoculum titre and represented the maximum possible number of recovered spores on a given test day. In addition, 0.1 ml of the spore inoculum was serially diluted in 0.1% Tween 80 solution and immediately plated (incubation at 37±2° C. for 16±2 hrs) to quantify inoculum spore titre on the day of coupon inoculation.

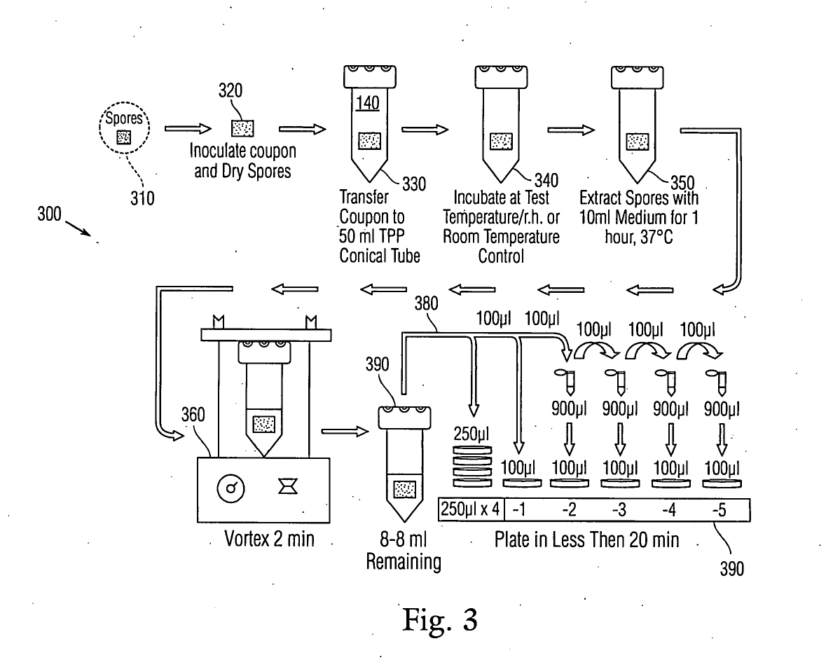

FIG. 3 shows a step-by-step diagram view 300 of the exemplary hot, humid air decontamination method. Spores 310 and another five replicates of each substrate were inoculated with B. thuringiensis Al Hakam spores. A total of ten dried coupons 320 for each coupon type and five coupon types yielded fifty test coupons. The coupons were transferred to a 50 ml TPP® conical tube 140 in a transfer process 330 and subject to incubation 340 at a test temperature and relative humidity. In an extraction process 350 spores were retrieved with a 10 ml medium for one hour at 37° C. A spin process 360 subjects the extracted spores to vortex for 2 minutes. After removal with 8.8 ml of medium remaining, the spores are cataloged in a segregation process 380. The spores are subdivided by plating at specified volume levels, each at 20 minutes.

Fifty coupons with dried spores, ten polypropylene tubes with dried spores and ten wet spore controls gave a total of seventy test substrates per test run. An identical set of seventy substrates were inoculated and held at room temperature during each test run. Thus, seventy test substrates and seventy room temperature substrates for a total of hundred-forty substrates were processed during each test run. In addition, a single non-inoculated coupon for each coupon type was incubated at room temperature to show that coupons were not contaminated.

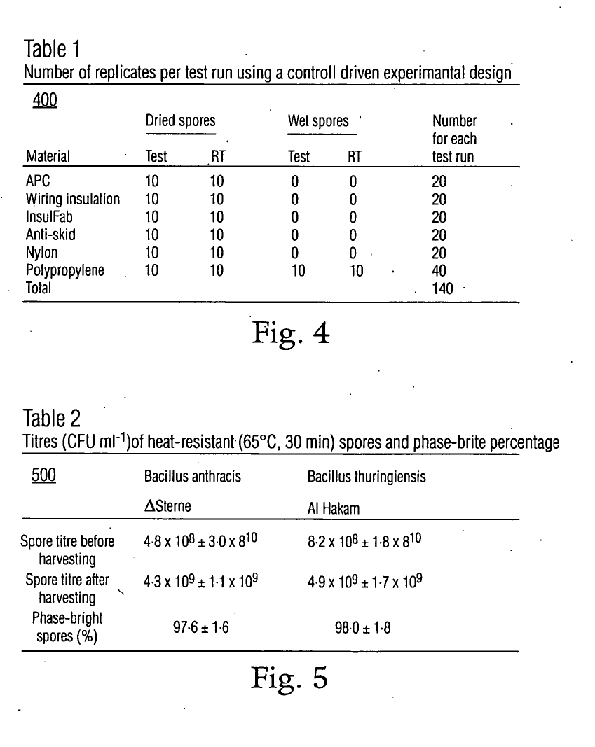

FIG. 4 shows a tabular view 400 of Table 1 with the Number of test replicates per test run using a control driven experimental design. Each test run is defined as a single combination of temperature, time and relative humidity that was tested within a single time interval. A substrate is defined as an individual coupon, polypropylene or wet sample. A replicate is defined as a substrate within a single test run which was inoculated with spores from an independent spore preparation. For each test run, five replicates of each substrate were inoculated with B. anthracis ΔSterne. The column identifies various materials for the substrate. Most of these materials were used to subject dry spores, and polypropylene was also employed for wet spores.

Quantification of spore survival: After exposure to specified temperature, relative humidity and time, spores were extracted from substrates and quantified for viability using dilution plating on tryptic soy agar (TSA) from Hardy Diagnostics of Santa Maria, Calif. An extraction medium was prepared, in particular: 3% tryptic soy broth (TSB) as T8907 from Fluka in Buchs, Switzerland; 0.25% buffered peptone water (BPW) as 1.07228.0500 from EMD in Darmstadt, Germany; 0.05% Tween 80 at pH 7 as BP338 from Fisher in Pittsburgh. Ten milliliterrs of preheated (37° C.) was added to each conical tube with dried spores (polypropylene) and incubated at 37° C., 30 min for spore extraction during the first iteration of testing. During the second iteration, 10 nil of preheated (37° C.) extraction medium, including 1% glucose (G5767) from Sigma in St Louis, Mo.; 3% TSB, 0.25% BPW, 0.05% Tween 80 at pH 7, was added to each conical tube and incubated at 37° C., 60 min for spore extraction. In both iterations, 5 ml of 2× extraction medium was added to the wet spore controls for a total of 10 ml and then incubated at 37° C.

Following incubation, conical tubes were vortexed for 2 min, 22±3° C. on a Glasco vortexer at a setting of 70. Samples were then serially diluted and plated on TSA within 20 min of vortexing. The total time from the addition of extraction medium to plating was <60 min during the first iteration of testing and <90 min during the second iteration of testing. Plates were scored for growth after incubation at 37±2° C. for 16±2 hrs. The conical tubes with the remaining 8.8 ml extraction medium, substrates and spores were also incubated at 37±2° C. for 16±2 hrs to qualitatively assess the viability of all remaining spores, including those not removed from substrates. If there were no colonies on the plates and no growth in conical tubes, then the survival was scored as zero. If there was growth evident in the conical tube but no colonies observed on the plates, then this was scored as 0.1 CFU ml⁻¹ because at least one viable spore had to be present for growth in the 8.8 ml of medium.

Spore survival calculations Wet spore controls, incubated at 22±3° C. and ambient relative humidity, served as the 100% recovery reference values for calculating spore survival after hot, humid air treatment and analysis. The number of spores extracted from each spore extraction control coupon (i.e., spores dried on substrates and incubated at 22±3° C. and ambient relative humidity) was divided by the number from the wet spore controls to calculate spore extraction percentage. The number of surviving spores of colony forming units (CFU) in ml⁻¹ from each hot, humid air-treated test substrate was then divided by the extraction percentage to determine the number of surviving spores in CFU m1⁻¹. This spore concentration was then multiplied by 10 ml to give a total number of spores surviving (CFU) for each test sample. A log₁₀ transformation of the total surviving spores was performed as log₁₀ (total CFU+1).

Transmission electron microscopy (TEM) analysis of untreated spores, heat-killed spores, pH-adjusted bleach-killed spores and outgrowing spores was conducted with B. anthracis ΔSterne, B. thuringiensis Al Hakam, B. cereus ATCC 4342 and B. atrophaeus ATCC 9372 spores to provide data for simulant selection and justification. Greater than 109 spores were prepared for TEM as previously described (Anon 2007; Buhr et al. 2008). Spores were inactivated by suspending in water and incubating at 82° C. for 48 hrs, or via treatment with pH-adjusted bleach as previously described (Anon 2007; Buhr et al. 2008): Spores were also inoculated at 5×10⁷ spores ml⁻¹ and incubated in Luria-Bertani broth at 37° C. and collected at various times up to 2 hrs to assess germination. The heat-killed and pH 7-adjusted bleach-treated spores were checked to confirm that none survived using dilution plating on TSA.

Results Strain selection: Preliminary tests with hot, humid air showed comparable spore kill kinetics among virulent B. anthracis Ames, attenuated B. anthracis ΔSterne and B. thuringiensis Al Hakam spores. Bacillus cereus ATCC 4342 spores showed higher survival rates, particularly for the wet spore controls (data not shown). This screening led to down-selection of B. anthracis ΔSterne and B. thuringiensis Al Hakam spores.

FIG. 5 shows a tabular view 500 of Table 2 Spore Preparation and Characterization. Each independent spore preparation surpassed the requirements of >10⁸ heat-resistant spores ml⁻¹ of sporulation medium and >95% purity by phase-contrast microscopy.

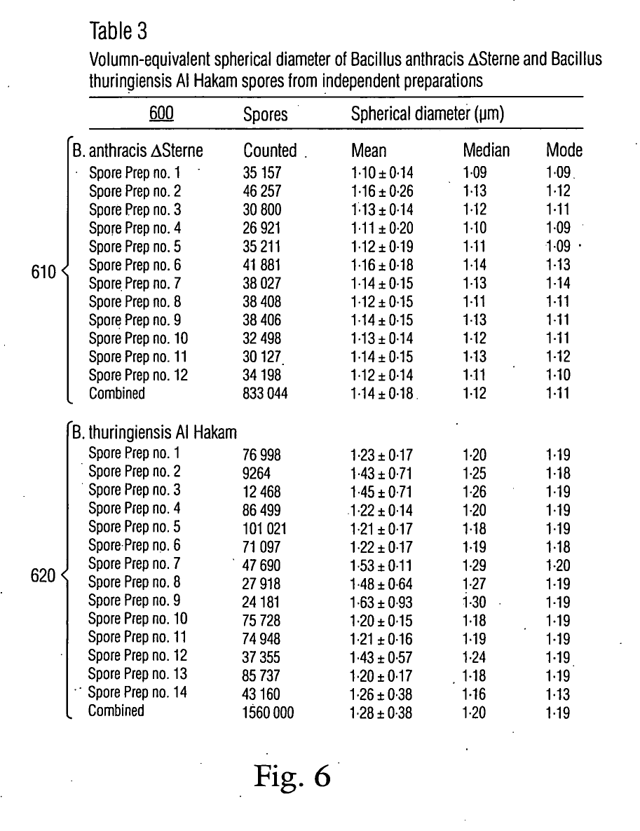

FIG. 6 shows a tabular view 600 of Table 3 Volume-equivalent spherical diameter of Bacillus anthracis ΔSterne and Bacillus thuringiensis Al Hakam spores from independent preparations. Spore size distribution data obtained from Coulter analysis show reproducible populations of uniformly sized particles indicative of high purity spores. B. thuringiensis Al Hakam spores tended to agglomerate at concentrations above 10⁹ spores ml⁻¹ as indicated by a tail on Coulter graphs (data not shown). This was likely due to B. thuringiensis spore hydrophobicity (Doyle et al. 1984; Koshikawa et al. 1989; Husmark and Ronner 1990; Ronner et al. 1990; Faille et al. 2002).

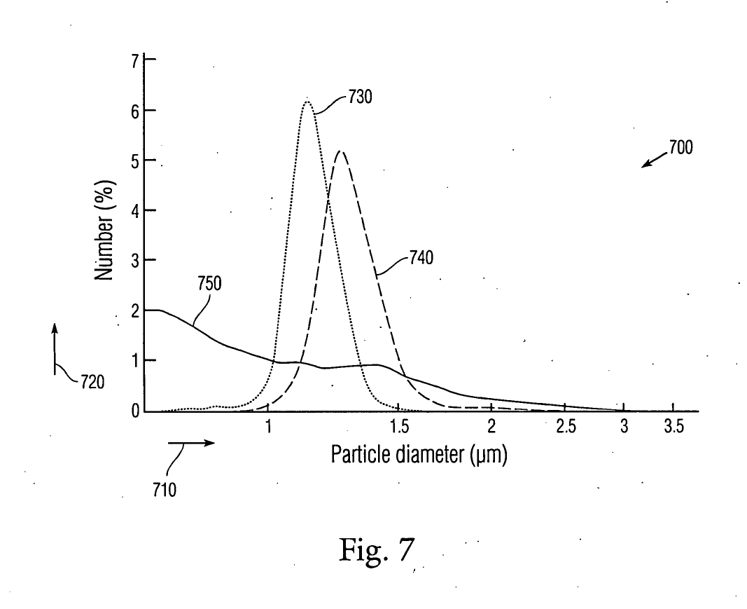

FIG. 7 shows a graphical plot view 700 of particle size distribution of three spore preparations. The abscissa 710 denotes particle diameter in μm, while the ordinate 720 denotes a normalized distribution for the percentage of particles of the corresponding size. These distributions represent 4295 total particles 730 of an in-house Bacillus anthracis ΔSterne preparation, 5,815 total. particles 740 of an in-house Bacillus thuringiensis Al Hakam preparation and 28,834 particles 750 of an independent laboratory's B. thuringiensis preparation. As can be observed, the Bacillus anthracis ΔSterne distribution 730 concentrates at about-6% at a particle diameter of about 1.1 μm whereas the Bacillus thuringiensis Al Hakam distribution 740 concentrates at about 5% at a particle diameter of 1.3 μm. The more generic B. thuringiensis distribution 750 displays a broader and less concentrated size profile.

View 700 shows Coulter analysis results for two in-house spore preparations and spores prepared by an independent laboratory for B. thuringiensis using different sporulation methods. Microscopy data supplied with the independent preparation indicated 95% phase-bright spores, a purity level that was not confirmed with in-house microscopy. The broad particle size distribution shown for the independent preparation suggested large quantities of debris, and dilution plating showed a viable spore concentration of 1.1×10⁸ CFU ml⁻¹ compared with a particle count of 2.3×10⁹ particles ml⁻¹ indicated by Coulter analysis. These data supported the use of Coulter analysis as an objective method for quantifying spore debris and purity.

Environmental test chamber set-up and validation Environmental chamber temperature and relative humidity recordings were collected from the built-in environmental chamber sensors as well as NIST-certified hygrometers placed on the inside of test TPP® conical tubes. Temperature and relative humidity were measured inside conical tubes with TPP® 0.2 μm filter-caps 140 and solid-capped conical tubes (data not shown). In all experimental conditions, temperature and relativity humidity measured by the hygrometer inside the TPP® conical tubes with filter-caps (but not those with solid caps) reached the environmental chamber set-point values within 40 min, and the set conditions were maintained until the experiment was terminated. Thus, the 0.2 μm-filtered caps 140 permitted sufficient moisture and heat exchange between the chamber and the inside of the conical tube.

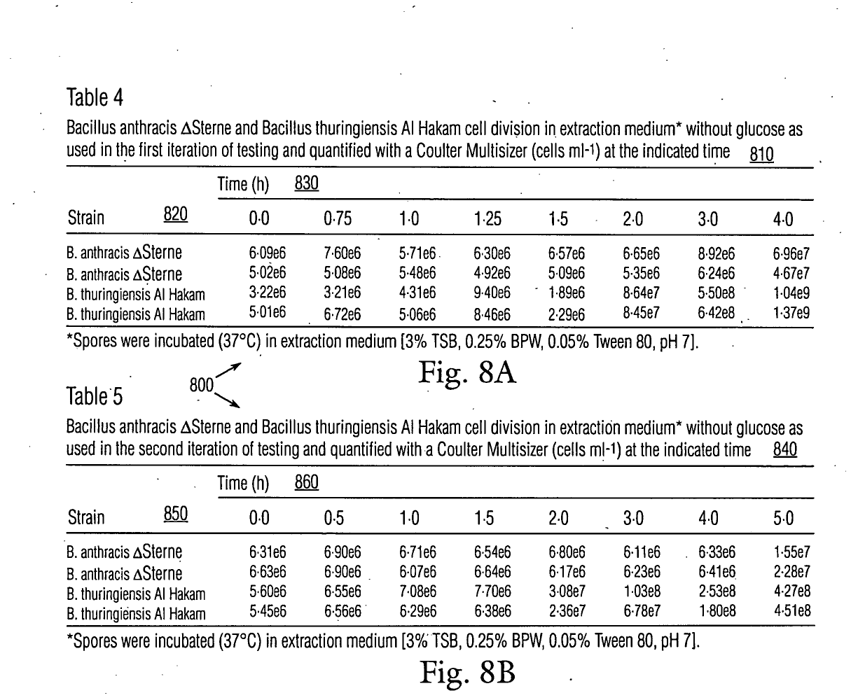

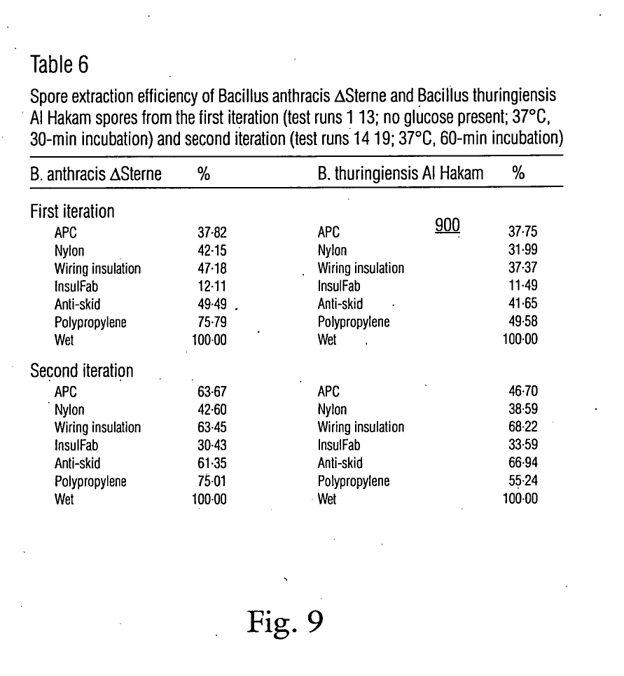

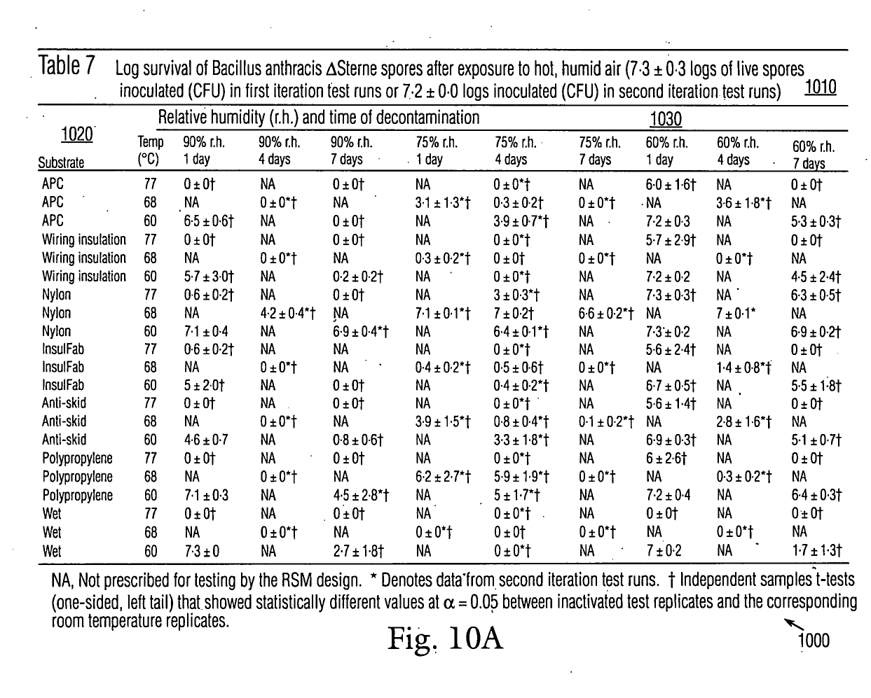

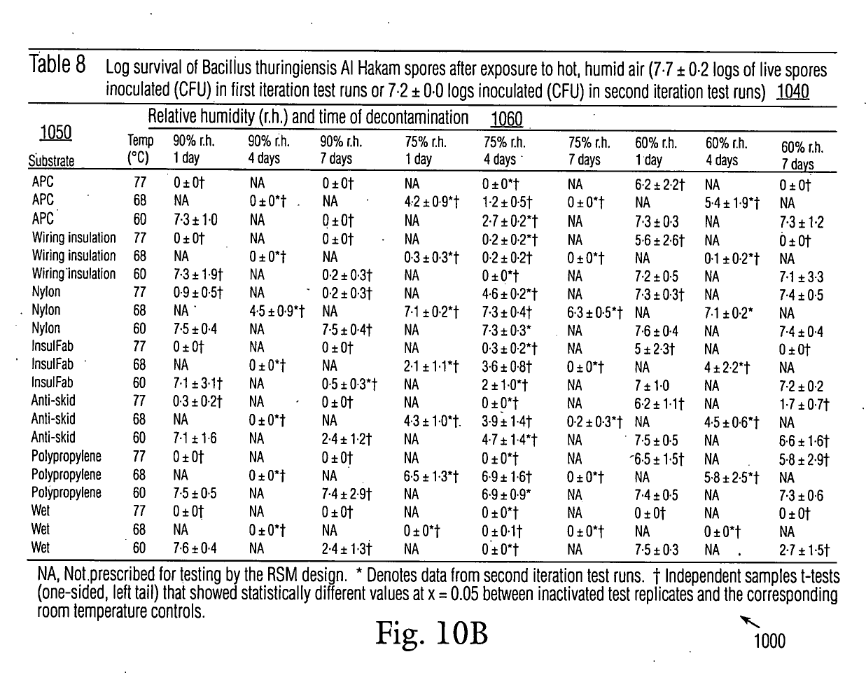

FIGS. 8A and 8B present tabular views 800 of Tables 4 and 5 cell division of B. anthracis ΔSterne and B. thuringiensis Al Hakam in an extraction medium. FIG. 8A shows a tabular view 810 listing the Bacillus variety in the column 820 and the row of times 830 for the array of cell quantities, in the medium without glucose. FIG. 8B shows a tabular view 840 of listing the Bacillus variety in the column 850 and the row of times 850 for the array of cell quantities in the medium with glucose. FIG. 9 shows a tabular view 900 of Table 6 Spore Extraction Efficiency of spores from first and second iteration runs. FIGS. 10A and 10B provide tabular views 1000 of Log Survival of Bacillus spores after exposure to hot, humid air. FIG. 10A shows a tabular view 1010 of Table 7 Log Survival of B. anthracis ΔSterne with the column 1020 listing substrates and the row 1030 providing the relative humidity and exposure duration. FIG. 10B shows a tabular view 1040 of Table 8 Log Survival of B. thuringiensis Al Hakam with the column 1050 listing substrates and the row 1060 providing relative humidity and exposure duration.

Spore extraction from substrates can be described as follows. A single-step extraction protocol that could be used for either B. anthracis ΔSterne or B. thuringiensis Al Hakam spores for all substrates was developed. Spore extraction with non-nutrient solutions (0.1% Tween 80 and 1% morpholinopropane-sulfonic acid, pH 7) was highly variable on nylon and InsulFab (data not shown). In order to improve spore extraction efficiency, a nutrient-rich medium was used for spore extraction, similar to products from standardized methods ASTM E2414-05, ASTM E-2197-02, AOAC 966.04 and AOAC 2008.05 (Anon 2002, 2005; Tomasino and Hamilton 2006; Tomasino et al. 2008). The timing of cell division was monitored after extraction medium was added to B. thuringiensis Al Hakam and B. anthracis ΔSterne spores to ensure that spores extracted from substrates were plated before cell division.

The number of B. thuringiensis Al Hakam cells began to increase at 1.25 hrs, while B. anthracis ΔSterne cell division was not observed until 4 hrs after the addition of extraction medium as indicated in view 810 (FIG. 8A) at Table 4. The faster germination and growth kinetics of B. thuringiensis Al Hakam determined that spores should be plated within one hour after the addition of extraction medium, prior to cell replication.

Because the RSM process was iterative, further optimization of the extraction process was possible. For the second test iteration, glucose was added to the extraction medium as a known catabolite repressor. Addition of glucose delayed measurable cell division of B. thuringiensis Al Hakam to 2 hrs after the addition of extraction medium as indicated in view 840 (FIG. 8B) at Table 5. This enabled the timing of the spore extraction to be extended by 30 min for the second iteration of testing. The increased spore extraction time generally improved spore extraction efficiency for most substrates as shown in view 900 at Table 6 Spore Extraction Efficiency in FIG. 9.

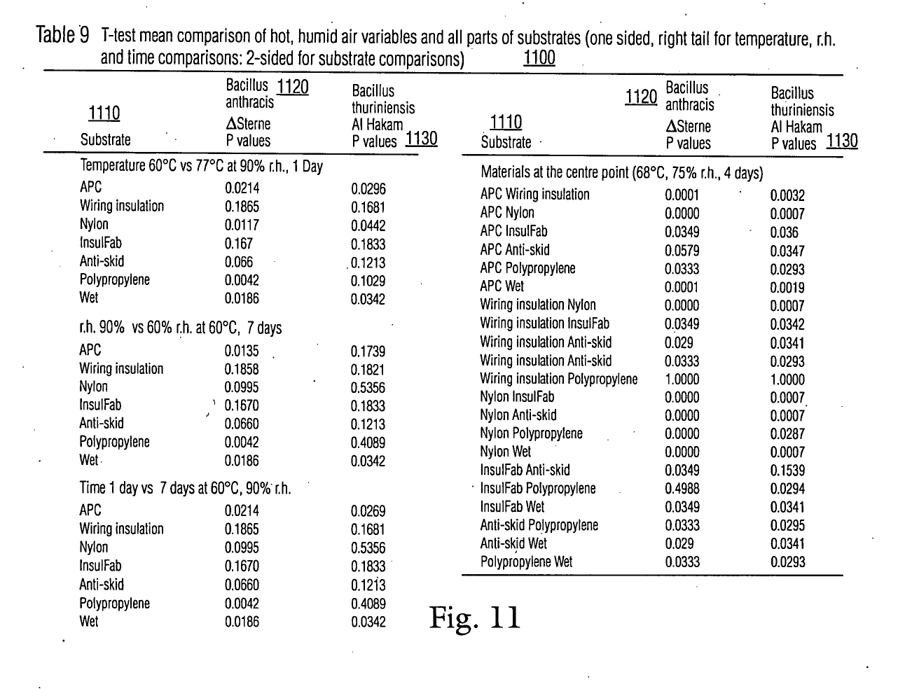

Hot, humid air decontamination can be demonstrated from the tabular information. FIGS. 10A and 10B illustrate tabular views 1000 of log survival of spores following hot, humid air decontamination in Tables 7 and 8. FIG. 10A shows a tabular view 1010 of Table 7 Log Survival of B. anthracis ΔSterne. The first column 1020 lists substrates and conditions, and the array 1030 provides the results for listed durations at specified relative humidity. FIG. 10B shows a tabular view 1040 of Table 8 Log Survival of B. thuringiensis Al Hakam. The first column 1050 lists substrates and conditions, and the array 1060 provides the results for listed durations at specified relative humidity. With the exception of the center point (68° C., 75% H_{R}, 4 days), the numbers represent the arithmetic mean of five replicates. Test reproducibility at the center point required the greatest level of confidence because partial inactivation of the spore population was anticipated. Hence, twenty-five replicates from five test runs were averaged per substrate for each strain at the center point test conditions. FIG. 11 shows a tabular view 1100 of Table 9 for t-test mean comparisons of hot, humid air variables and pairs of substrates. The tabular view 1100 displays columns for conditions with associate substrate 1110, P-values for B. anthracis ΔSterne 1120 and P-values for B. thuringiensis Al Hakam 1130.

The total number of spores per substrate were determined from room temperature controls and ranged between 7.1 and 7.8 logs of spores (data not shown). Based on the results of independent samples t-tests, the number of viable spores recovered after the mildest hot, humid air test run (60° C., 60% H_{R}, 1 day) was statistically identical (α=0.05) to the room temperature controls for 12 of 14 strain/substrate combinations. Spore survival for the majority of other combinations of strain/substrate/test run was statistically different compared to room temperature controls at α=0.05. Less than 1-log (10¹) of viable spores was recovered for either strain on any substrate after the harshest hot, humid air test run (77° C., 90% H_{R}, 7 days). Statistical t-tests indicated significant differences (α=0.20) in spore inactivation at higher temperature, higher relative humidity and longer incubation times for 36 of 42 comparisons shown in view 1100 at Table 9 in FIG. 11. A larger number of replicates would be needed to further increase the statistical confidence.

The influence of substrate surfaces on spore survival kinetics was seen in the comparison of spore survival rates on each substrate at the RSM center point (68° C., 75% H_{R}, 4 days). FIG. 11 illustrates a tabular view 1100 for Table 9 with a statistically significant difference (α=0.05) in spore survival for 18 of 21 substrate-to-substrate comparisons (B. anthracis ΔSterne) and 19 of 21 substrate-substrate comparisons (B. thuringiensis Al Hakam) at the center point. There was <0.5 logs of spore survival for spores dried on electrostatically charged wiring insulation and wet spore controls (i.e., spores suspended in aqueous 0.1% Tween 80). However, seven logs (7-log or 10⁷) of spores survived on nylon and more than five logs (>5-log or 10⁵) of spores survived on polypropylene (oil-based materials). Survival of spores dried onto anti-skid, APC and InsulFab was between these two extremes.

Spore survival results were remarkably similar for B. anthracis ΔSterne and B. thuringiensis Al Hakam, as shown in view 1000. There were some minor differences between species at the mildest test conditions (60° C., 90% H_{R}, 1 day and 60° C., 60% H_{R}, 1 day). There were no statistical differences in B. anthracis ΔSterne spore survival at 60° C., 90% H_{R}, 1 day on 3 of 7 substrates compared with the room temperature controls in view 1010 at Table 7 in FIG. 10A, and there were no statistical differences on 5 of 7 substrates at 60° C., 60% H_{R}, 1 day compared with the room temperature controls shown in view 1010 at Table 7 in FIG. 10A.

For B. thuringiensis Al Hakam spores, there was no statistical difference in 6 of 7 substrates for the same comparisons at 60° C., 90% H_{R}, 1 day and 7 of 7 substrates at 60° C., 60% H_{R}, 1 day shown in view 1040 at Table 8 in FIG. 10B. This suggested that B. thuringiensis Al Hakam spores survived slightly better on some substrates under the mildest test conditions. The most noticeable strain difference was at moderate hot, humid air conditions (68° C., 60% H_{R}, 4 days) on polypropylene. These conditions were effective at inactivating B. anthracis ΔSterne spores as shown in view 1010 at Table 7 in FIG. 10A, but not B. thuringiensis Al Hakam spores in view 1040 at Table 8 in FIG. 10B.

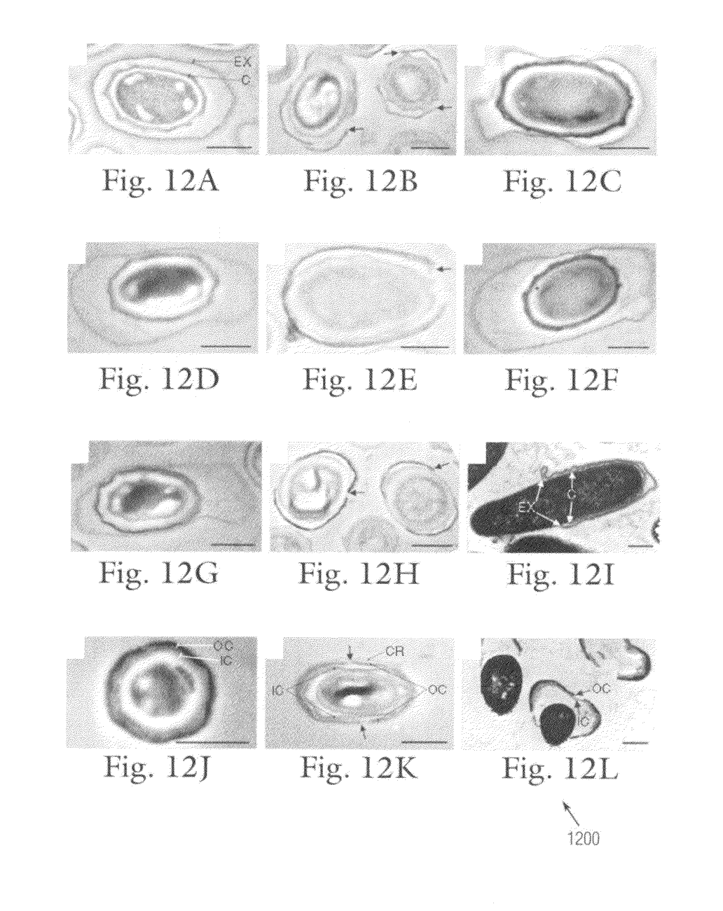

Transmission electron microscopy (TEM) of spores is provided by an array of photograph images in FIGS. 12A through 12L as view 1200. FIGS. 12A, 12B and 12C show B. anthracis ΔSterne. FIGS. 12D, 12E and 12F show B. thuringiensis Al Hakam. FIGS. 12G, 12H and 121 show B. cereus ATCC 4342. FIGS. 12J, 12K and 12L show B. atrophaeus ATCC 9372. Labeled reference abbreviations include C for coat, OC for outer coat, IC for inner coat, EX for exposporium, CR for crust. Arrows point to some breaks in the coat material in FIGS. 12B, 12E, 12H and 12K. Scale lines at bottom right corner indicate 0.5 μm. No obvious structural changes were observed in B. anthracis ΔSterne, B. thuringiensis Al Hakam, B. cereus (ATCC 4342) and B. atrophaeus (ATCC 9372) spores that were heat-killed as shown in the photographs of view 1200. Likewise, Coulter analysis showed no size changes in spores after heat inactivation (data not shown).

In contrast, the TEM images of spores treated with a chemical decontaminant for 15 min showed significant damage to the outer structures. The pH-adjusted bleachtreated macrobacillus spores were stripped of exosporia. The outer protein coat was damaged and often stripped as broken shards from the inner coat, which appeared intact. Exosporia and protein coats were less damaged after shorter 1-min treatments with pH-adjusted bleach (data not shown). All pH-adjusted bleach-treated B. atrophaeus spore images revealed gaps at the center of the outer coat, suggesting that the entire coat circumference was degraded at the longitudinal center. Both the inner coat and crust layers appeared intact around the B. atrophaeus spores, although all three outer layers were mostly separated from each other. Spore size measurements supported these observations: B. atrophaeus spore size was unchanged, while macrobacillus spores were smaller after pH-adjusted bleach treatment (Buhr et al. 2008; data not shown).

Another species comparison included TEMs of outgrown spores. B. cereus spore exosporium and coat layers were burst during germination and outgrowth, which was similar to B. anthracis (Steichen et al. 2007). Bacillus atrophaeus spore outgrowth was similar to that of B. subtilis (Santo and Doi 1974). Thus, species within the macrobacillus group manifested similar germination/outgrowth morphologies, and these differed from the microbacillus group. The single break in the exosporium and in each coat layer of outgrown B. cereus spores was markedly different than the disintegrated exosporium and broken shards of oxidizer-treated spores. Conversely, the oxidizer-generated gap in B. atrophaeus spores was observed only in the outer coat and appeared at the approximate location where the outer coat disintegrates during outgrowth.

Test method development represents a critical step towards developing and evaluating future decontaminants. This is especially true for mild technologies such as hot, humid air, where spore inactivation is explored at the limits of the decontamination technology. There has been a need in the current efforts to carefully control and reproduce data from test runs that partially inactivated spore populations. In order to increase data confidence, three test methods were developed: a spore preparation protocol useful for both B. thuringiensis and B. anthracis; a 0.2 μm filter-cap tube method for both testing of hot, humid air and spore extraction; and a control driven experimental design. The control driven experimental design included 140 substrates (not including sterile coupon controls) per test run. This was greater than ten times the recommended maximum of twelve samples per day for the AOAC 2008.05 decontamination test method (Tomasino et al. 2008; Buhr et al. 2011). The three methods were embedded within a statistical design of experiments, specifically RSM. In addition, spores were characterized by Coulter analysis and electron microscopy.

This disclosed effort has established test methods and satisfied the requirements for statistical design to show spore inactivation using hot, humid air decontamination. Such method advancements improve confidence in performance data, decrease test time, reduce test costs and support the transition of decontamination technologies from the laboratory to the field. The hot, humid air test methods were developed to translate to biosafety level three (BSL3) testing where safety and security requirements constrain test data output. This is highly applicable because existing and newly proposed regulations concerning possession or use of select agents or toxins limit the types of facilities and personnel that can perform BSL3 work (Anon 2011). The control-driven experimental design included 140 substrates (not including sterile coupon controls) per test run, or greater than ten times the recommended maximum of twelve samples per day for the AOAC 2008.05 decontamination test method (Tomasino et al. 2008).

The test methods and experimental design permitted the examination of spore inactivation in relationship to five variables: temperature, time, relative humidity, substrate and strain. Temperature, time and relative humidity are the three variables that can be controlled during hot, humid air decontamination. The conditions for those three variables were selected in order to explore the limits of spore inactivation for hot, humid air decontamination. The RSM directed experimental design also required three equally spaced conditions for each of the three decontamination variables. Test conditions were selected such that spore populations would survive the mildest conditions, be partially inactivated at the mid-point conditions and be fully inactivated at the harshest conditions. The data support the selection of the experimental conditions because spores were inactivated within the RSM-directed requirements.

End-user requirements will dictate the importance of each of the three decontamination variables on hot, humid air decontamination. For example, an end-user of sensitive equipment (electronics, aircraft, etc.) may have a different requirement for decontamination time compared with an end-user of a ground vehicle fleet. The RSM-directed experimental design permits a broad range of end-users to apply hot, humid air decontamination technology as adjustments can be made for the three critical decontamination variables based on the requirements set by the end-user.

Materials to be decontaminated can also influence the requirements set by an end-user. Spore inactivation on different substrates was significantly different at test conditions that were between the mildest and harshest test runs. The largest number of spores survived on nylon and polypropylene, while the fewest number of spores survived in solution (wet spores) and on wiring insulation. The exosporium is the outer spore structure that imparts hydrophobic characteristics, interacts with materials and likely impacted spore survival on different materials (Doyle et al. 1984; Koshikawa et al. 1989; Husmark and Ronner 1990; Ronner et al. 1990; Charlton et al. 1999; Todd et al. 2003; Redmond et al. 2004; Faille et al. 2002; Henriques and Moran 2007; Ball et al. 2008).

A hypothesis that may explain the differences in spore survival on different substrates was that porous substrates and hydrophobic, oil-based substrates repel water vapour. Thus, spore survival was highest on nylon because it was porous and hydrophobic, while spore survival on wiring insulation was lowest because this substrate had electrostatic properties. The exclusion of water vapor may have reduced heat transfer to the spores and/or reduced any effects of dissociated water molecules (H₃O⁺ and OH⁻). These hypothesized effects may be particularly pronounced at higher temperatures because the dissociation constant of water is more than 20-fold greater at 70° C. than at 20° C. (Fernandez-Prini et al. 2004).

Selection and justification of simulants is needed to achieve meaningful results and to accumulate sufficient data for confidence in a new technology, particularly for field testing (Buhr et al. 2012). Bacillus thuringiensis offers numerous advantages as a B. anthracis simulant over other candidate species (Greenberg et al. 2010). The Al Hakam strain of B. thuringiensis is genetically closer to B. anthracis than many other B. thuringiensis strains, has a fully sequenced genome and possesses no known toxin genes, and it was the suspected simulant from the Iraqi Al Hakam weapons-of-mass-destruction programme (Radnedge et al. 2003; Challacombe et al. 2007). Bacillus thuringiensis pesticidal strains (e.g., kurstaki) are commercially manufactured to favour the production of entomotoxin (Beegle and Yamamoto 1992; AvignoneRossa and Mignone 1995).

The crystal toxin, debris and fillers in such preparations vary in type and quantity to create a set of unknown variables that could impact decontamination kinetics and data interpretation. Bacillus thuringiensis Al Hakam is similar to B. anthracis because neither strain produces crystal toxin during sporulation. Bacillus thuringiensis Al Hakam spores survived similarly or slightly better than B. anthracis ΔSterne spores under the same hot, humid air conditions on the various substrates.

In total, these data support the justification of B. thuringiensis Al Hakam spores as a stimulant, as there is a preference that a simulant be slightly more difficult to inactivate than the agent in order to mitigate the risk of insufficient decontamination. Conversely, the use of B. thuringiensis Al Hakam may be less critical for evaluating more aggressive decontamination technologies. For example, inactivation kinetics for B. thuringiensis Al Hakam spores were similar to that of B. subtilis spores under the traumatic conditions generated by a shock-wave reaching temperatures above 226° C. (McCartt et al. 2011).

Coulter and TEM analysis showed morphological similarities among macrobacillus spores before decontamination, after decontamination and during spore outgrowth. Spores inactivated with hot, humid air remained intact with no noticeable changes to spore ultra-structure. The analyses revealed no obvious clues regarding the target(s) of hot, humid air. In contrast, morphological damage was seen in the outer layers of spores, particularly macrobacillus spores, after treatment with pH-adjusted bleach. Bleach is known to be a strong oxidizer with general chemical reactivity and high heat is a denaturant. These data support the idea that bleach has pleiotropic effects and damages multiple spore surface targets; that hot, humid air is a mild decontaminant relative to pH-adjusted bleach; and that macrobacillus spores are more appropriate simulants for B. anthracis. Comparison of relevant material surfaces treated with both decontaminants would be required to confirm this assessment for end-users.

Characterization of spore morphology before decontamination, after decontamination and during spore outgrowth also supports the identification of potential spore targets for both future decontamination development and characterization of the assembly of spore structures. The macrobacillus species show very similar mechanisms of exosporium and coat shedding during outgrowth that is visibly distinct from the microbacilli species (Steichen et al. 2007; Santo and Doi 1974). The morphological damage to the exosporia and outer coat layers of pH-adjusted bleach-treated macrobacillus spores also contrasted with the specific morphological target in the outer coat of B. atrophaeus spores. Despite the differences in the outer layers of spores from macrobacillus and microbacillus species, the inner coat and cortex of all species remained intact. This suggests some common chemistry among all species that holds the coat shards together in the dormant spore. This chemistry is currently being analyzed to further elucidate the mechanisms of coat assembly and disassembly.

The disclosure established baseline performance data for hot, humid air decontamination on clean substrates contaminated with highly purified and characterized spores. Many variables may have a significant impact on spore inactivation results. For example, differences in spore preparation conditions and spore recovery have been shown to influence the results of heat resistance testing (Alderton and Snell 1969; Cazemier et al. 2001; Melly et al. 2002). A future objective is to characterize the impact of debris on spore inactivation kinetics where the quantities and types of debris can be combined with high-purity spores under controlled conditions, thereby treating debris content as an independent, known variable (Buhr et al. 2011, 2012).

Response surface modeling for hot, humid air decontamination of materials contaminated with Response surface methodology (RSM) for Bacillus anthracis ΔSterne and Bacillus thuringiensis Al Hakam spores using a face-centered cube design (FCD) was used to describe and predict spore inactivation of Bacillus anthracis ΔSterne and B. thuringiensis Al Hakam spores after exposure to hot, humid based on a recently published decontamination data set.

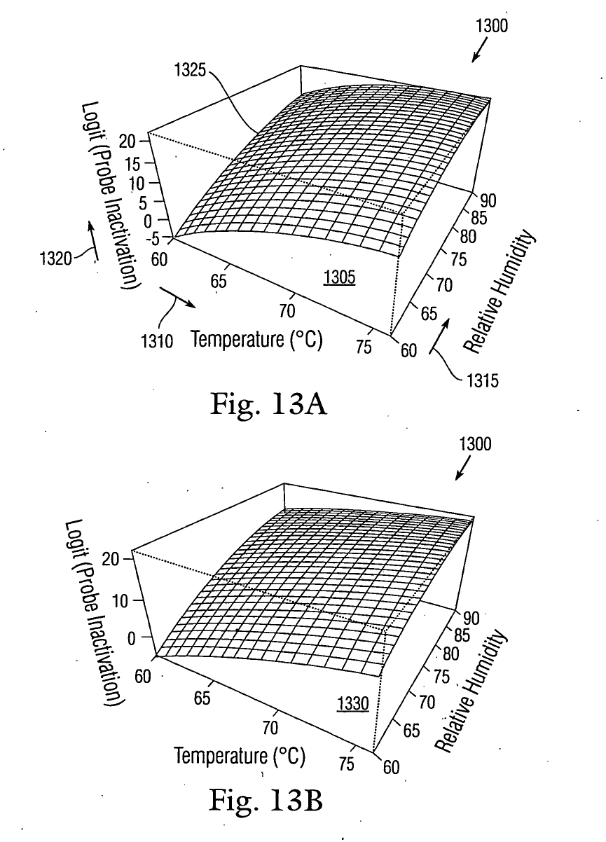

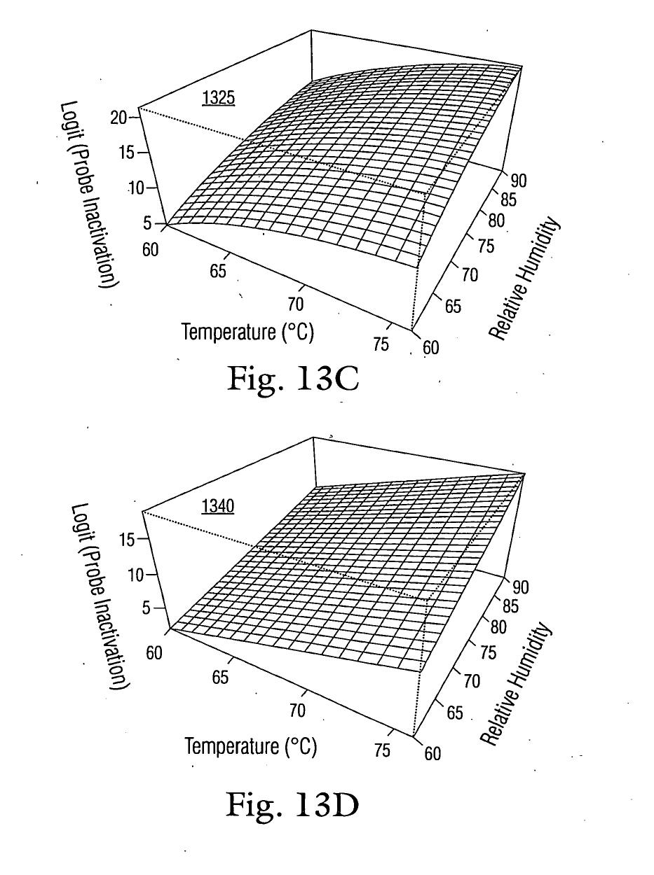

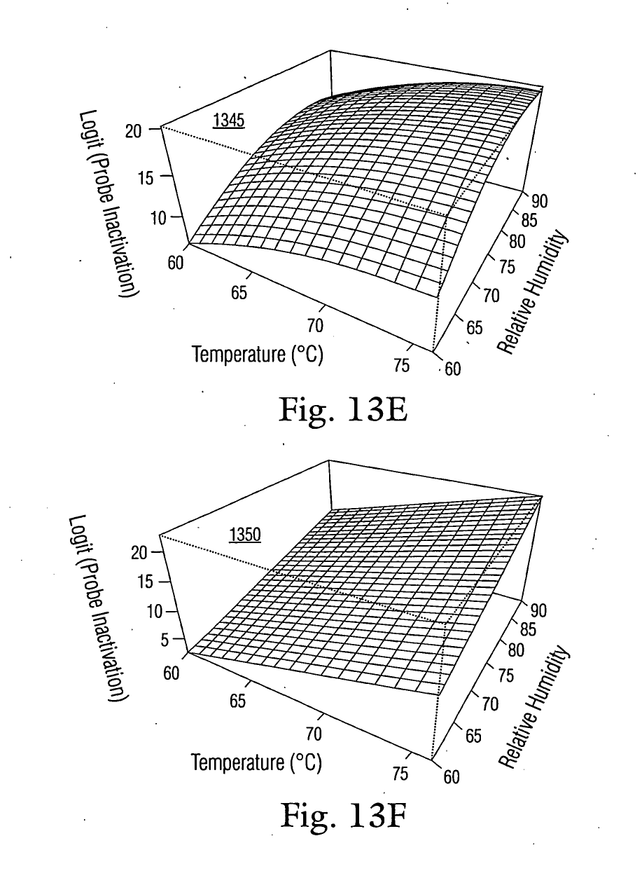

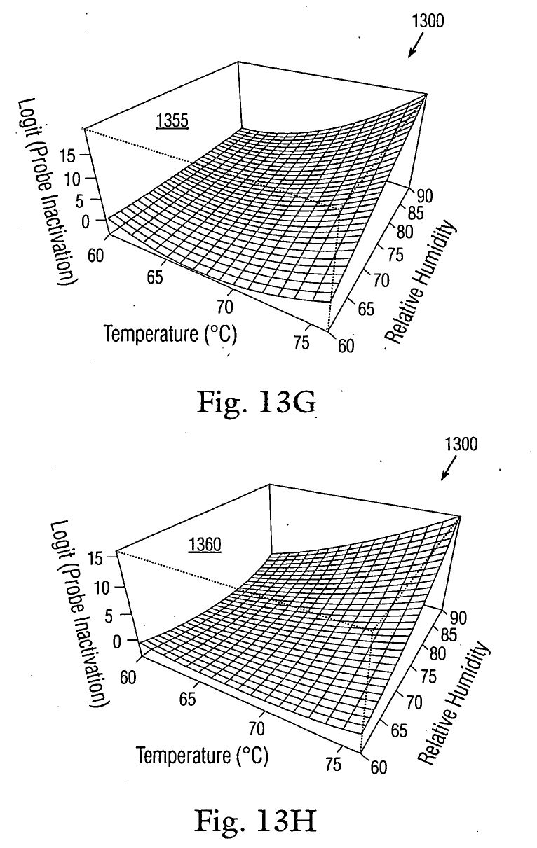

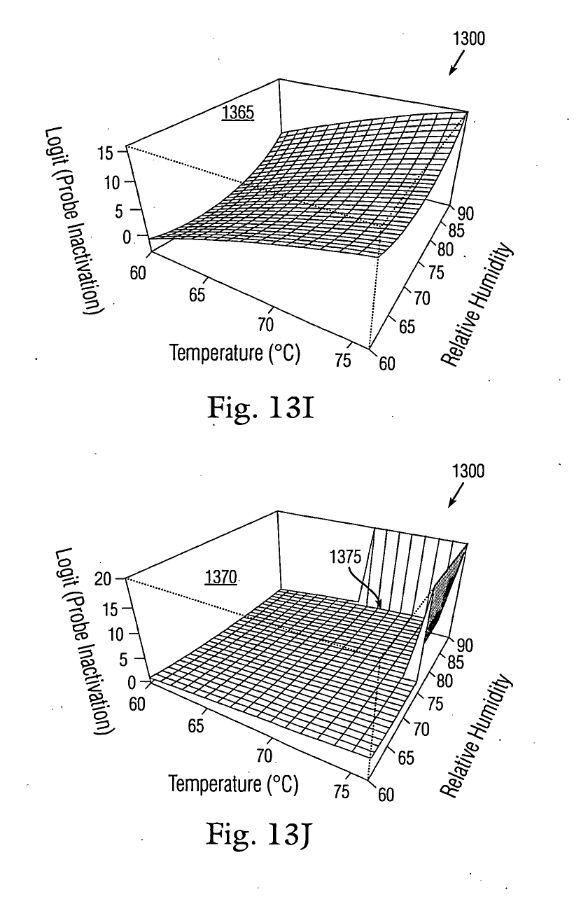

FIGS. 13A through 13J illustrate perspective three-dimensional (3D) response surface views 1300 of spore survivals. FIG. 13A shows a response surface view 1305 of B. anthracis ΔSterne on APC for four days. The abscissa 1310 (left downward) denotes temperature (independent condition); the ordinate 1315 (right upward) denotes relative humidity (independent condition); and the azimuth 1320 (left upward) denotes logit of probable inactivity (dependent variable). FIG. 13B shows a response surface view 1320 of B. thuringiensis Al Hakam on APC for four days. FIG. 13C shows a response surface view 1335 of B. anthracis ΔSterne on antiskid for four days. FIG. 13D shows a response surface view 1340 of B. thuringiensis Al Hakam on antiskid for four days. FIG. 13E shows a response surface view 1345 of B. anthracis ΔSterne on InsulFab for four days. FIG. 13F shows a response surface view 1350 of B. thuringiensis Al Hakam on InsulFab for four days.

FIG. 13G shows a response surface view 1355 of B. anthracis ΔSterne on InsulFab for four days. FIG. 13H shows a response surface view 1360 of B. thuringiensis Al Hakam on nylon for four days. FIG. 13I shows a response surface view 1365 of B. anthracis ΔSterne on polypropylene for four days. FIG. 13J shows a response surface view 1370 of B. thuringiensis Al Hakam with a sharp boundary 1375 of logit shift upward towards extreme humidity and temperature. These surface plots are described further herein.

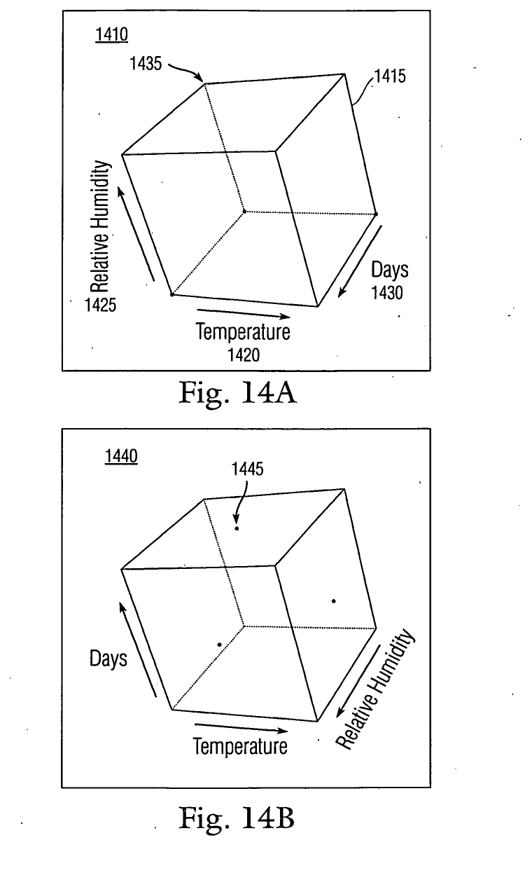

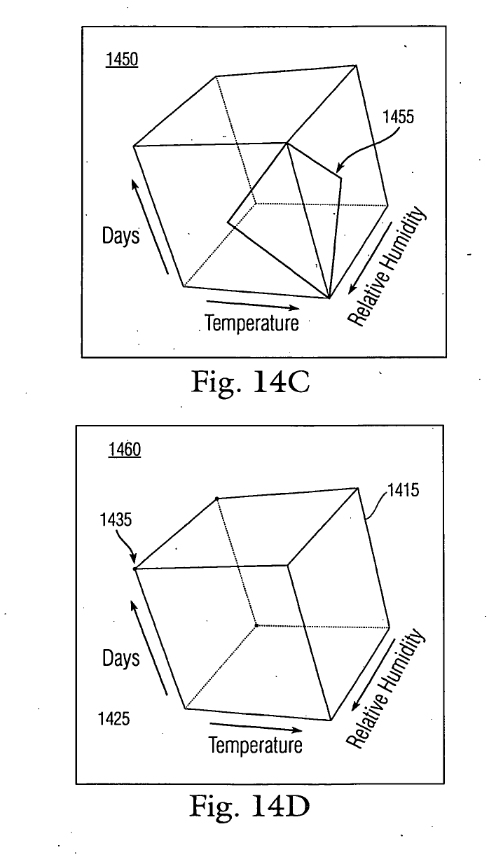

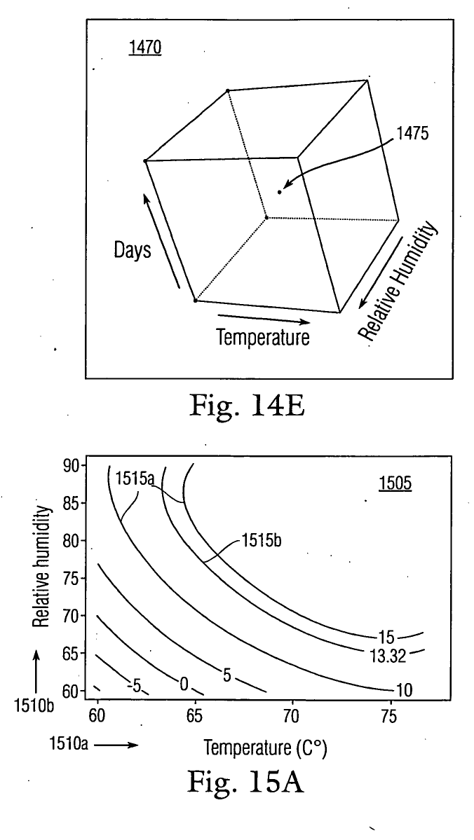

FIGS. 14A through 14E displays perspective views 1400 of inactivation diagrams related to the RSM parameter box view 200. FIG. 14A shows a perspective view 1410 of a parameter box 1415 for B. anthracis ΔSterne. The abscissa 1420 (rightward) denotes temperature; the ordinate 1425 (upward) denotes relative humidity; and the azimuth 1430 (downward) denotes exposure time, and points 1435 identify conditions at which spore survival points survive on wet solution, with all other conditions having complete inaction. FIG. 14B shows a perspective view 1440 of the design space box for B. thuringiensis Al Hakam survival points 1445 on propylene. FIG. 14C shows a perspective view 1450 of the design space box for B. anthracis ΔSterne with an inactivation outline 1455. FIG. 14D shows a perspective view 1460 of B. anthracis ΔSterne with survival spore points 1465. FIG. 14E shows a perspective view 1470 of B. thuringiensis Al Hakam with survival spore points 1475. The points correspond to some of the center and face positions 250 of the matrix of conditions that were tested.

For each strain-substrate pair, an attempt was made to fit a first or second order model. All three independent predictor variables (temperature, relative humidity, and time) were significant in the models except for B. thuringiensis Al Hakam on nylon where time was not significant: Modeling was unsuccessful for wiring insulation and wet spores, cases with complete spore inactivation in the majority of the experimental space. In cases where a predictive equation could be fit, example response surface plots with time set to four days were generated. The survival of highly purified Bacillus spores can be predicted for most materials tested if given the settings for temperature, relative humidity, and time. Dependence of spore survival and predictability of this survival on material substrate has been demonstrated by the exemplary process.

Hot, humid air decontamination is being explored at the limits of sporicidal efficacy as a decontamination technology with improved materials compatibility. This disclosure focuses on describing the application of RSM to experimental design and data analysis in the hot, humid air studies. The objective herein is to mathematically describe the effectiveness of hot, humid air decontamination, thus enabling an end-user to find acceptable operating conditions depending on the material to be decontaminated.

Hot, humid air decontamination data have been analyzed from experimental results. The RSM represents a statistical procedure used to understand, improve, and optimize a process. The objective is to model a response variable in terms of one or more independent predictor variables. Spore survival data was used to calculate the log odds of inactivation for each test conditions. Second order polynomial models are most commonly used in RSM because they are flexible and their parameters are easy to estimate. An exemplary relation, including a blocking term is employed for the disclosed protocol. In particular, this generalized expression can be written as:

$$\begin{matrix}y = ln ( \frac{p ( x )}{1 - p ( x )} ) \\ = {β}_{0} + {\sum }_{j = 1}^{k} {β}_{j} {x}_{j} + {\sum }_{j = 1}^{k} {β}_{jj} {x}_{j}^{2} + {\sum }_{i < j}^{ } {\sum }_{j = 2}^{k} {β}_{ij} {x}_{i} {x}_{j} + b ,\end{matrix} \qquad ( 1 )$$

where y is the dependent parameter quantifying long odds spore inactivation, xⱼ represents a set of independent variables, βⱼ denotes a correlation coefficient from j=0, 1, . . . k, and b represents the blocking term.

The log odds of spore inactivation is the logit, i.e., the natural logarithm of

$$\frac{p ( x )}{1 - p ( x )}$$

where p(x) is the probability of inactivation calculated from raw data as expressed by:

$$p ( x ) = 1 - \frac{{n}_{s}}{{n}_{t}} , \qquad ( 2 )$$

such that nₛ is the number of surviving spores and nₜ is the number of spores tested. This relation represents an example of analysis of variance (ANOVA) for comparing sensitivity to change driving factors.

RSM is typically used sequentially. An experiment is first done to screen potential predictor variables. For current settings of the predictors being near an optimum, supplementary runs are done to fit a second order model. If the experimental region is not near an optimum, the method of steepest ascent can be used to find the optimum or arrive at a region of operability should an optimum be unattainable. In other situations, the region of operability is the entire experimental region. Such was the case for this study. The design employed the face-centered cube (FCD) in this scenario, as shown in view 200, to represent the environmental domain.

Consumers of spore inactivation information are frequently interested in achieving a 6-log (10⁶) reduction in contamination. This translates into a probability of spore inactivation equal to 0.999999 or a log odds of spore inactivation equal to

$$log ( \frac{0.999999}{1 - 0.999999} ) = 13.81551 .$$

To be conservative, the 90% lower confidence surface was used to estimate the probability of inactivation for the contour plots. In cases where a model could be fit using eqn. (1), a contour plot for temperature versus relative humidity is given in original units with days fixed at four.

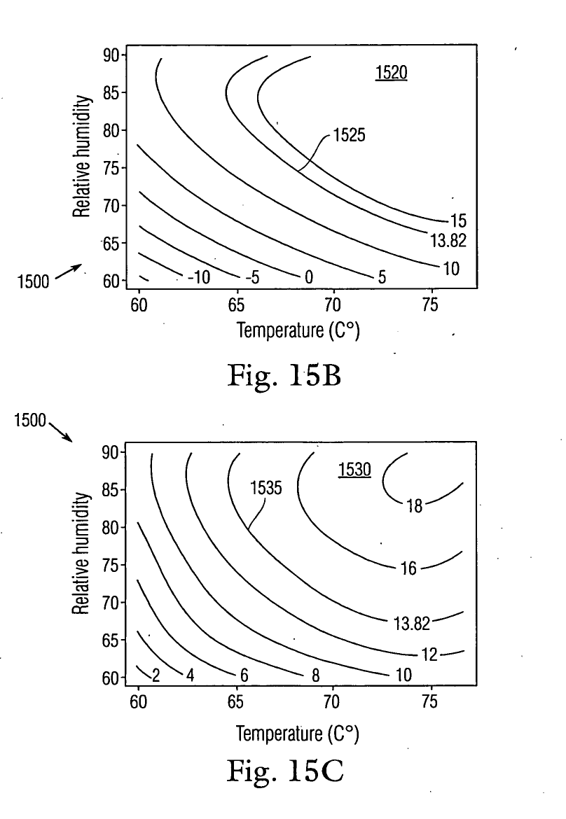

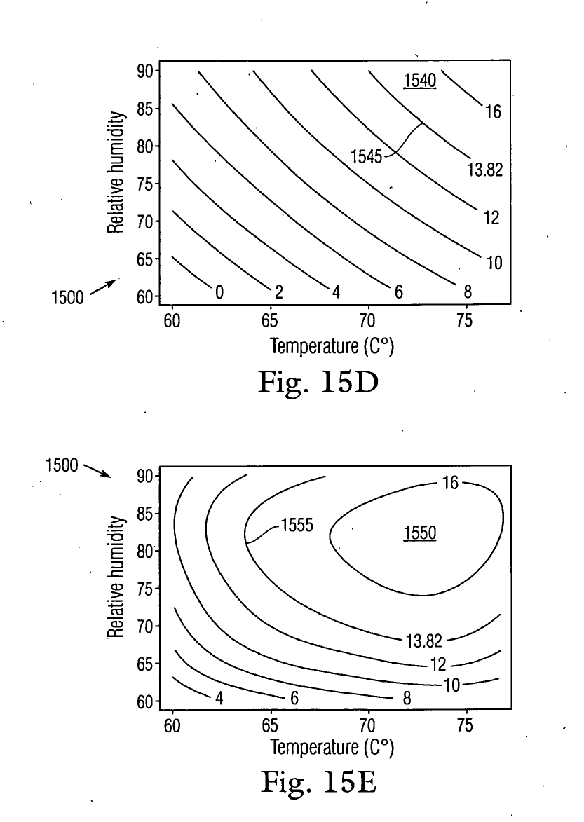

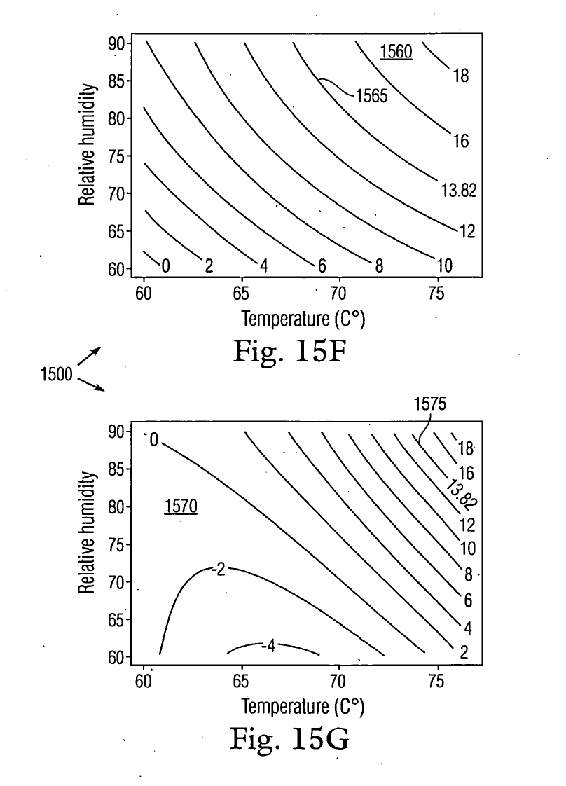

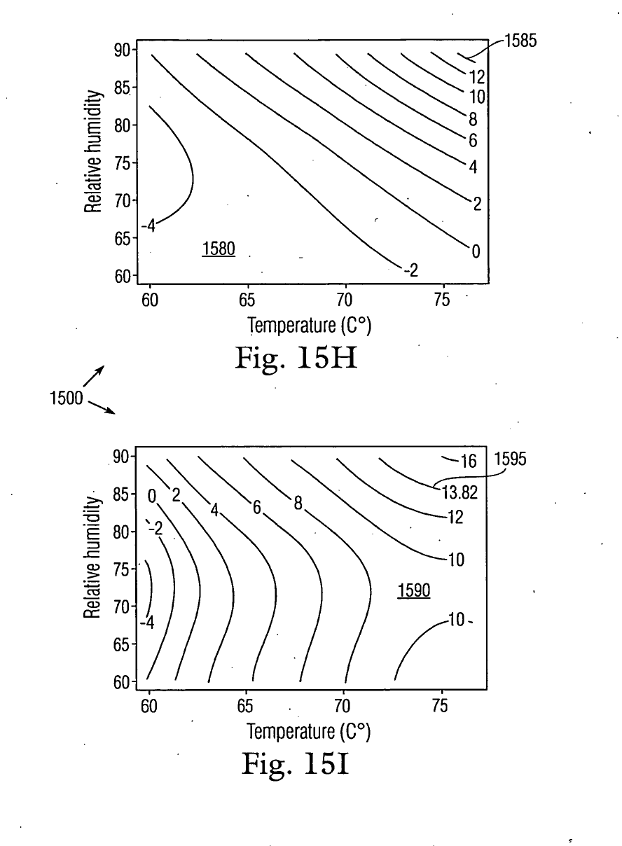

FIGS. 15A through 151 illustrates two-dimensional (2D) contour plot views 1500 of the 90% LCL surface for log odds of spore inactivation at four days. FIG. 15A shows a contour plot 1505 for B. anthracis ΔSterne on APC. The abscissa 1510a denotes temperature while the ordinate 1510b denotes relative humidity for conditions. Contour curves 1515a indicate inactivation logit values, and curve 1515b identifies the 6-log value. Similarly, FIG. 15B shows a contour plot 1520 for B. thuringiensis Al Hakam on APC with curve 1525 denoting the 6-log value. FIG. 15C shows a contour plot 1530 for B. anthracis ΔSterne on antiskid with curve 1535 denoting the 6-log value. FIG. 15D shows a contour plot 1540 for B. thuringiensis Al Hakam on antiskid with curve 1545 denoting the 6-log value.

Similarly, FIG. 15E shows a contour plot 1550 for B. anthracis ΔSterne on InsulFab with curve 1555 denoting the 6-log value. FIG. 15F shows a contour plot 1560 for B. thuringiensis Al Hakam on InsulFab with curve 1565 denoting the 6-log value. FIG. 15G shows a contour plot 1570 for B. anthracis ΔSterne on nylon with curve 1575 denoting the 6-log value. FIG. 15H shows a contour plot 1580 for B. thuringiensis Al Hakam on nylon with curve 1585 denoting the 6-log value. FIG. 15I shows a contour plot 1590 for B. anthracis ΔSterne on polypropylene with curve 1595 denoting the 6-log value.

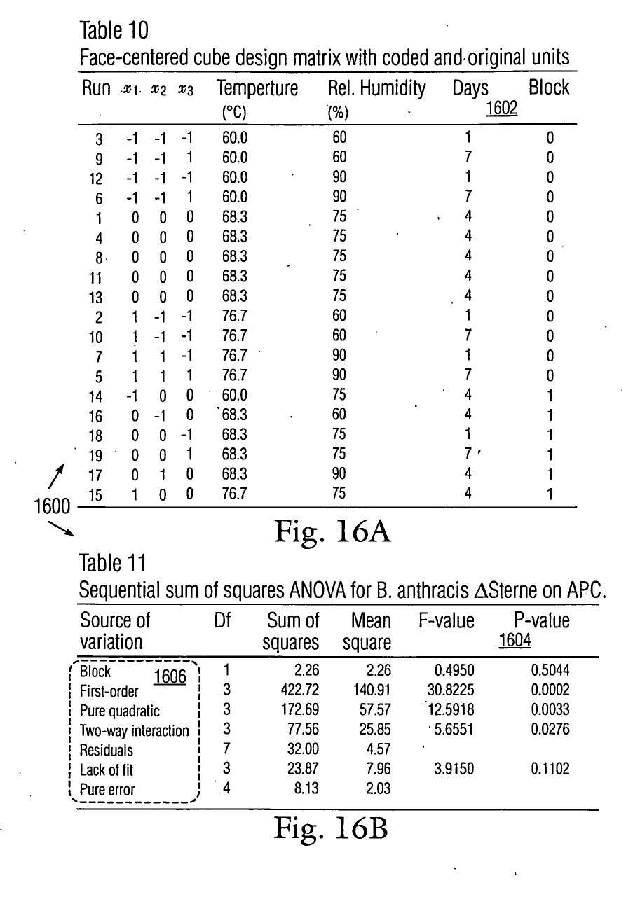

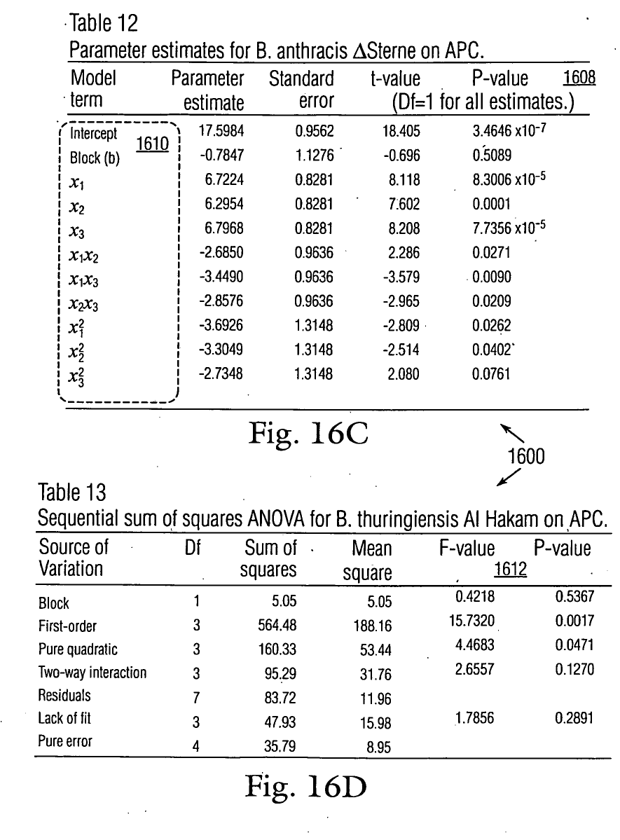

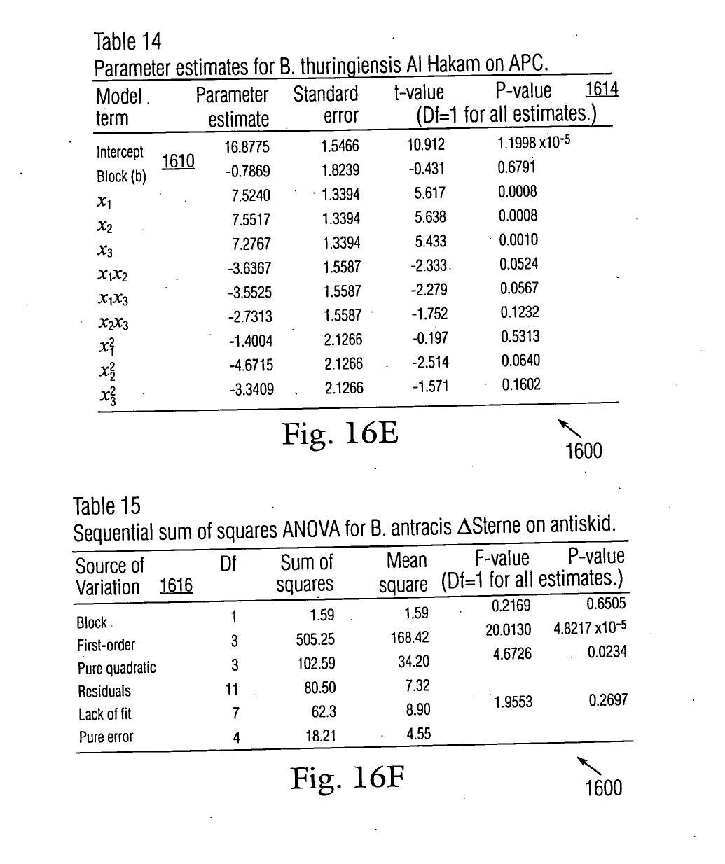

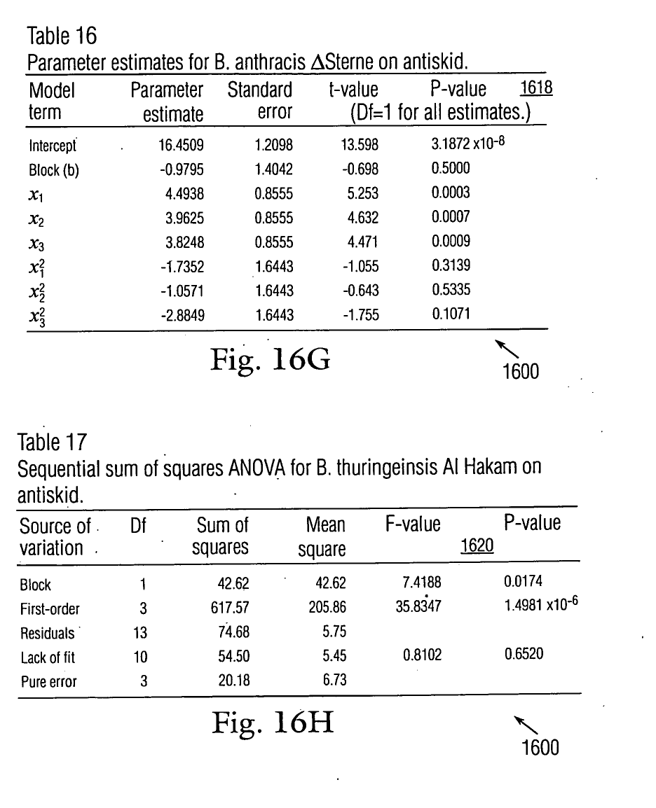

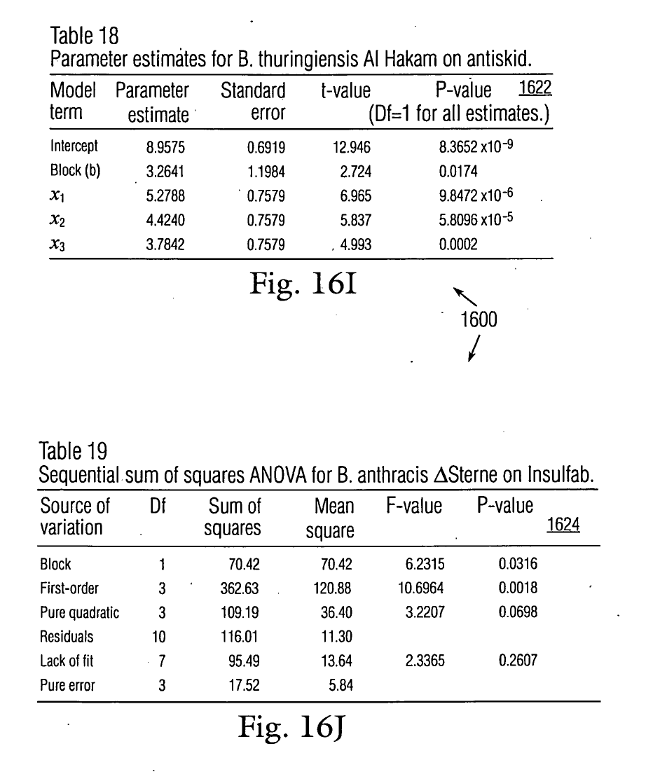

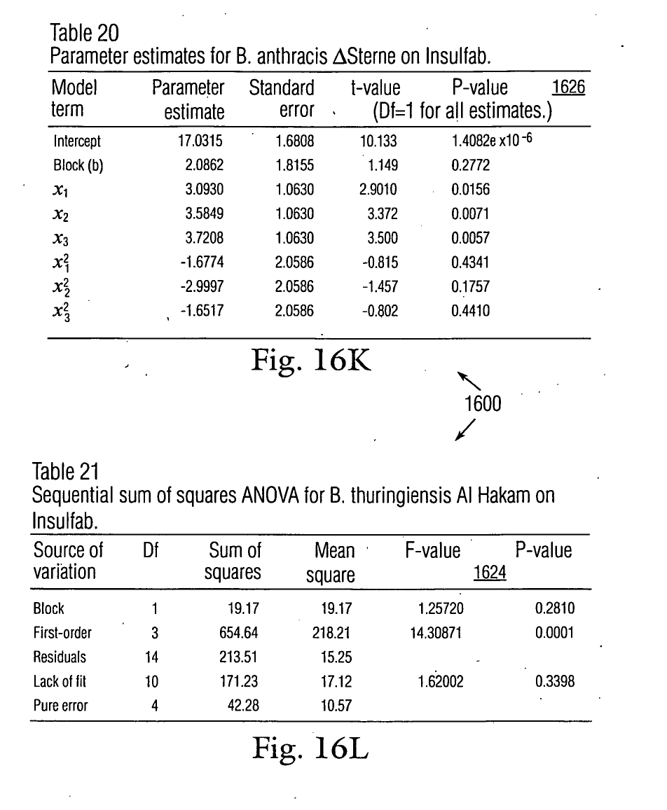

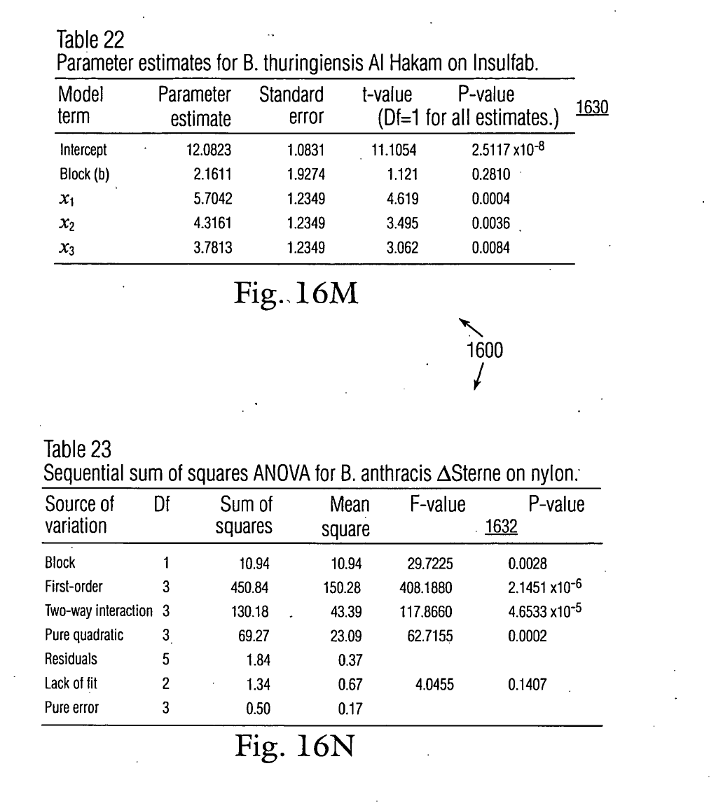

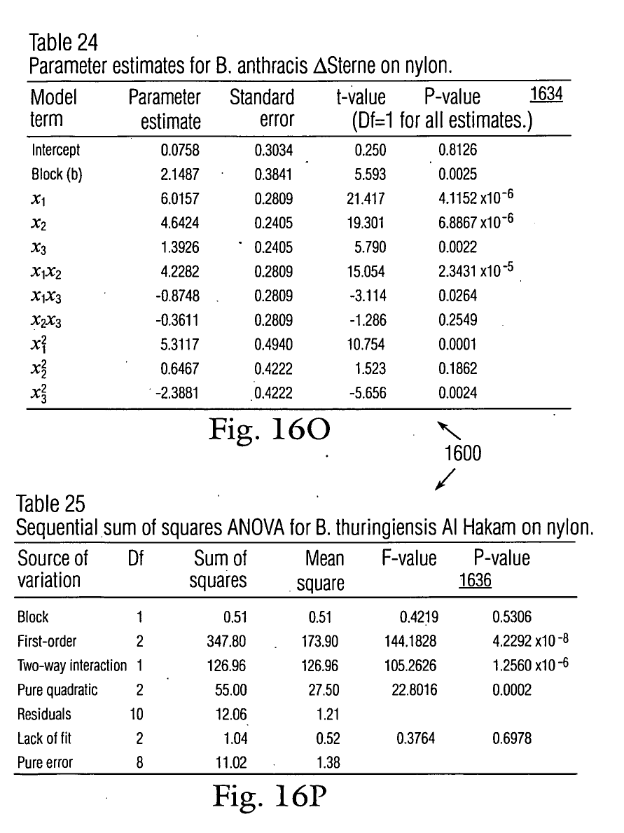

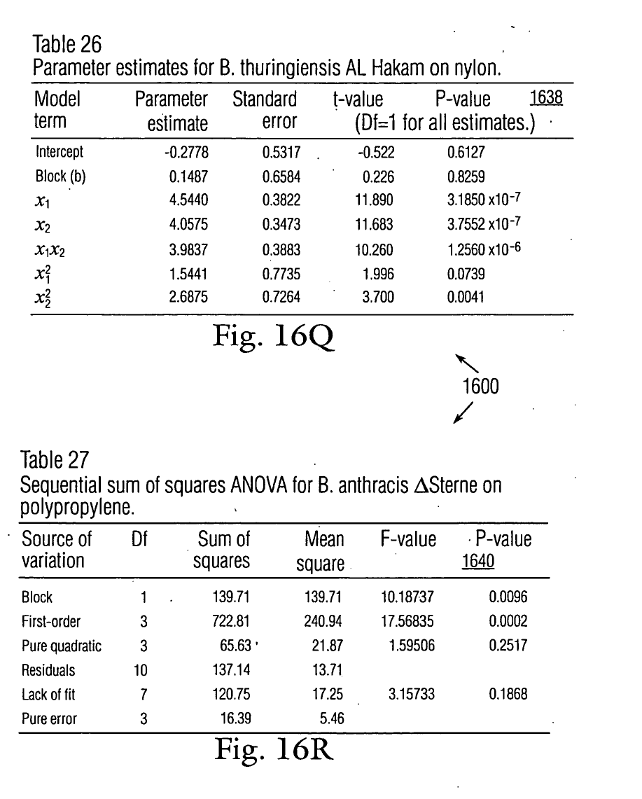

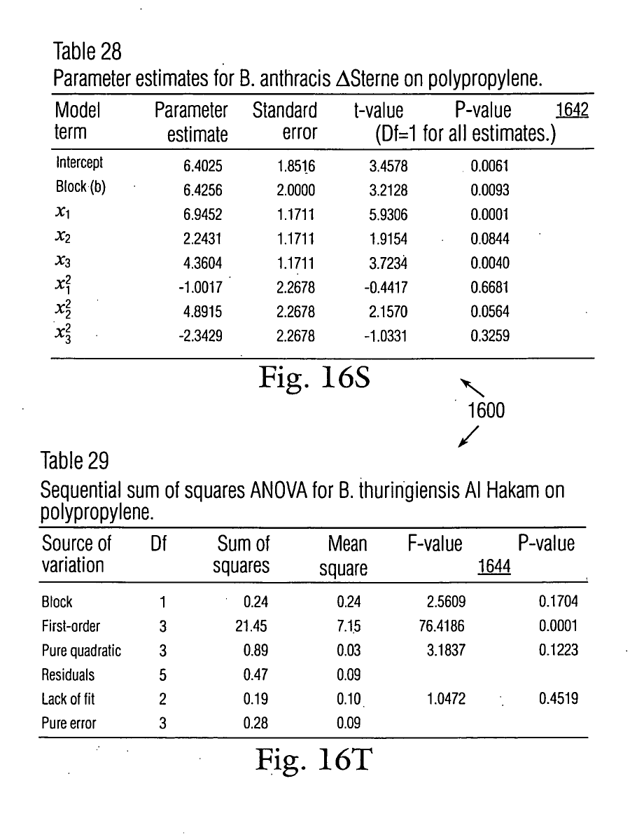

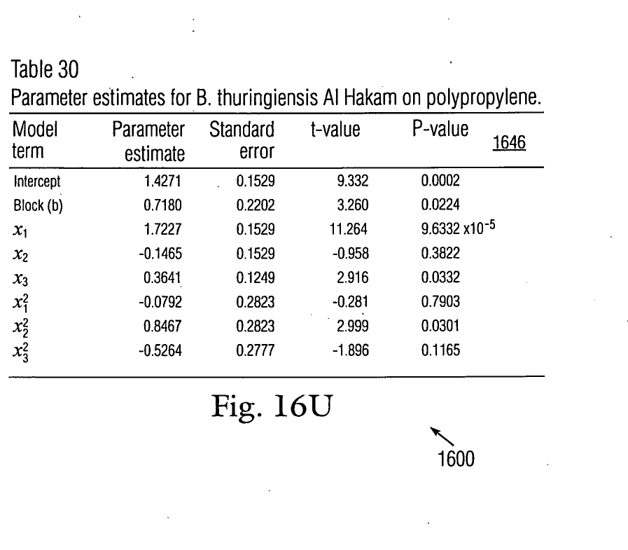

FIGS. 16A through 16U show tabular views 1600 of parameters used to evaluate the data. FIG. 16A shows tabular view 1602 with Table 10 Face-centered cube design matrix in terms of coded and original predictor variables. FIG. 16B shows tabular view 1604 with Table 11 for Sequential Sum of Squares ANOVA for B. anthracis ΔSterne on aircraft performance coating (APC) with the source of results variation 1606 as a column. FIG. 16C shows tabular view 1608 with Table 12 Parameter estimates for B. anthracis ΔSterne with model terms 1610 as a variable column. FIG. 16D shows tabular view 1612 with Table 13 Sequential Sum of Squares ANOVA for B. thuringiensis Al Hakam on APC. FIG. 16E shows tabular view 1614 with Table 14 Parameter estimates for B. thuringiensis Al Hakam on APC.

FIG. 16F shows tabular view 1616 with Table 15 Sequential Sum of Squares ANOVA for B. anthracis ΔSterne on antiskid. FIG. 16G shows tabular view 1618 with Table 16 Parameter estimates for B. anthracis ΔSterne on antiskid. FIG. 16H shows tabular view 1620 with Table 17 Sequential Sum of Squares ANOVA for B. thuringiensis Al Hakam on antiskid. FIG. 16I shows tabular view 1622 with Table 18 Parameter Estimates for B. thuringiensis Al Hakam on antiskid.

FIG. 16J shows tabular view 1624 with Table 19 Sequential Sum of Squares ANOVA for B. anthracis ΔSterne on InsulFab. FIG. 16K shows tabular view 1626 with Table 20 Parameter estimates for B. anthracis ΔSterne on InsulFab. FIG. 16L shows tabular view 1628 with Table 21 Sequential Sum of Squares ANOVA for B. thuringiensis Al Hakam on InsulFab. FIG. 16M shows tabular view 1630 with Table 22 Parameter estimates for B. thuringiensis Al Hakam on InsulFab.

FIG. 16N shows tabular view 1632 with Table 23 Sequential Sum of Squares ANOVA for B. anthracis ΔSterne on nylon. FIG. 16O shows tabular view 1634 with Table 24 Parameter estimates for B. anthracis ΔSterne on nylon. FIG. 16P shows tabular view 1636 with Table 25 Sequential Sum of Squares ANOVA for B. thuringiensis Al Hakam on nylon. FIG. 16Q shows tabular view 1638 with Table 26 Parameter estimates for B. thuringiensis Al Hakam on nylon.

FIG. 16R shows tabular view 1640 with Table 27 Sequential Sum of Squares ANOVA for B. anthracis ΔSterne on polypropylene. FIG. 16S shows tabular view 1642 with Table 28 Parameter estimates for B. anthracis ΔSterne on polypropylene. FIG. 16T shows tabular view 1644 with Table 29 Sequential Sum of Squares ANOVA for B. thuringiensis Al Hakam on polypropylene. FIG. 16U shows tabular view 1646 with Table 30 Parameter estimates for B. thuringiensis Al Hakam on polypropylene.

The measured proportion response p(x) was the basis for the RSM analysis. The proportion of spores inactivated defined as one minus the number of surviving spores divided by the average number of spores tested. For use in RSM analyses, the predictors were standardized to coded variables (x₁, X₂, and x₃) using

$${x}_{1} = \frac{T - 155}{15}$$

where T is the temperature in degrees Fahrenheit (° F.) converted from

$${x}_{1} = \frac{T - 68. \overline{3}}{8. \overline{3}}$$

for temperature in degrees Celcius (° C.), relative humidity (H_{R}) using

$${x}_{2} = \frac{{H}_{R} - 75}{15}$$

where H_{R} is percent relative humidity, and time using

$${x}_{3} = \frac{D - 4}{3}$$

where D is time in days (Montgomery 2009, Myers et al. 2009, Lenth 2009, Hosmer & Lemeshow 2000, Agresti 1996, Agresti 2002). Each run in this test matrix was conducted using five independent preparations of each spore strain. Further justification for selection of temperature, relative humidity and time variables regarding surface decontamination is provided by Buhr et al. in “Hot, humid air decontamination of materials contaminated with Bacillus anthracis ΔSterne and B. thuringiensis Al Hakam spores”, J. Appl Microbiol 113, 1037-1051 (2012).

As view 1602 at Table 10 shows, there were three predictors, temperature (T), relative humidity (H_{R}), and time in days (D). These were transformed to the coded variables x₁, x₂, and x₃ in order to better determine the relative size of their effects and described above. The measured response of p(x₁, x₂, x₃) constitutes the proportion of spores inactivated divided by the average number of spores tested. Because the response is a proportion, the proper link function is the logit (i.e., log odds) as observed in eqn. (1). If a particular material had complete spore inactivation on one or two observations, the proportion was conservatively imputed as the minimum detection level at “0.1 average number of spores tested” to bound the logit away from approaching infinity and avoid an undefined value. In some cases, however, too many coupons yielded complete inactivation and could not be modeled using this relation.

An attempt to fit a second order model was made for each strain on each material. ANOVA tables for the sequential sum of squares were used to determine which group of terms contributed significantly to the models. The groups were first-order terms (x₁, x₂, x₃), pure quadratic terms (x₁², x₂², x₃²), and interaction terms (x₁x₂, x₁x₃, x₂x₃). For a group of terms being deemed significant, all terms from that group were retained in the model. Each model included a blocking variable b because the observations were gathered in two phases: the first phase for the factorial and center points, the second phase for the face-centered points. Information concerning individual parameter estimates, standard error and statistical significance is provided in separate ANOVA tables. In cases where a model could not be reasonably fit, an illustration showing where there was complete inactivation (or lack thereof) is provided.

Each model was subjected to diagnostic measures consisting of the F-test for lack of fit, examination of raw residuals and jack-knifed residuals as compared to fitted values, and examination of normal plots based on both raw residuals and jackknifed residuals. These diagnostics were crucial for outlier detection and in determining for which cases a model could not be fit. In the left hand side of the equations below, x is the vector (x₁, x₂, x₃) for proportion response p(x).

All test variables were at the lowest settings during test run three, and there was no or minimal spore inactivation. This observation could not be modeled using eqn. (1). Hence, data from test run three were omitted, and the model was refitted. The ANOVA table for Sequential sum of squares in view 1604 at Table 11 in FIG. 16B shows that a second order model with pure quadratic terms and two-way interactions was needed. The F-test for lack of fit has a P-value of 0.110, which is sufficiently large so as not to reject the model. Correlation parameters R² and adjusted-R² for are model are 0.9548 and 0.8901, respectively.

View 1608 at Table 12 in FIG. 16C shows the parameter estimates, standard errors, t-values, and P-values for B. anthracis ΔSterne on APC. All second order terms are significant for this scenario. The fitted equation for inactivation of B. anthracis ΔSterne on APC is:

y_{Ba(1)}=17.5984−0.7847b+6.7224x₁+6.2954x₂+6.7968x₃−2.6850x₁x₂−3.4990x₁x₃−2.8576x₂x₃−3.6923x₁²−3.3049x₂²−2.7348x₃²′ (3)

where y_{Ba(1)} is the log odds of spore inactivation for the B. anthracis on the first APC material. View 1305 in FIG. 13A shows a slice of the response surface at four days and blocking term b fixed at its mean value of 0.3333.

All test variables for B. thuringiensis Al Hakam on APC were at the lowest settings during test run three, and there was no or minimal spore inactivation. This observation could not be modeled using eqn. (1). Hence, data from test run three were omitted, and the model was refitted. The ANOVA table for Sequential sum of squares in view 1612 at Table 13 in FIG. 16D shows a second order model with pure quadratic terms was needed and should probably include two-way interactions as well. The F-test for lack of fit has a P-value of 0.289, which does not indicate lack of fit. Correlation parameters R² and adjusted-R² for the model are 0.9079 and 0.7763, respectively.

View 1614 at Table 14 at FIG. 16E lists the parameter estimates, standard errors, t-values, and P-values. The fitted equation for inactivation of B. thuringiensis Al Hakam on APC is:

y_{Bt(1)}=16.8775−0.7869b+7.5240x₁+7.5517x₂+7.2767x₃−3.6367x₁x₂−3.5525x₁x₃−2.7313x₂x₃−1.4004x₁²−4.6715x₂²−3.3409x₃²′ (4)

where Y_{Bt(1)} is the log odds of spore inactivation on the first APC material. View 1330 in FIG. 13B shows a slice of the response surface at four days and blocking term b fixed at its mean value of 0.3333.

The ANOVA table for Sequential sum of squares in view 900 at Table 6 in FIG. 9 shows that a second order model with pure quadratic terms was needed for B. anthracis ΔSterne on antiskid. Two-way interaction terms did not contribute significantly to the model (p=0.846) and were not included. The F-test for lack of fit has a P-value of 0.270, which does not indicate lack of fit. Correlation parameters R² and adjusted-R² for the model are 0.8833 and 0.8091, respectively.

View 1010 at Table 7 at FIG. 10A shows the parameter estimates, standard errors, t-values, and P-values. The pure quadratic term for days is the significant second order term. The fitted equation for inactivation of B. anthracis ΔSterne on antiskid is:

y_{Ba(2)}=16.4509−0.9795b+4.4938x₁+3.9625x₂+3.8248x₃−1.7352x₁²−1.0571x₂²−2.8849x₃² (5)

where Y_{Ba(2)} is the log odds of spore inactivation on the second antiskid material. View 1335 in FIG. 13C shows a slice of the response surface at four days and blocking term b fixed at its mean value of 0.3158.

Run number one for B. thuringiensis Al Hakam on antiskid, a center point, was difficult to model and a clear outlier in the residual and normal plots. This run was omitted and the model refitted. The ANOVA table for Sequential sum of squares in view 1620 at Table 17 in FIG. 16H shows that a first order model was needed. Neither two-way interactions nor pure quadratic effects were significant (p=0.560 and p=0.513, respectively). The F-test for lack of fit has a P-value of 0.652, which does not indicate lack of fit. Correlation parameters R² and adjusted-R² for the model are 0.8984 and 0.8671, respectively.

View 1622 at Table 18 in FIG. 16I lists the parameter estimates, standard errors, t-values, and P-values. The fitted equation for inactivation of B. thuringiensis Al Hakam on antiskid is

y_{Bt(2)}=8.9575+8.2641b+5.2788x₁+4.4240x₂+3.7842x₃, (5)

where Y_{Bt(2)} is the log odds of spore inactivation on the second material. View 1340 FIG. 13D shows a slice of the response surface at four days and blocking term b fixed at its mean value of 0.3333.

Run number one for B. anthracis ΔSterne on InsulFab, a center run (within the environment box), was deemed an outlier and hence was excluded from the analysis. The ANOVA table for Sequential sum of squares in view 1624 at Table 19 in FIG. 16J shows that a second order model with pure quadratic terms should be considered. Two-way interaction terms did not contribute significantly to the model (p=0.890) and were not included. The F-test for lack of fit has a P-value of 0.261, which does not indicate lack of fit. Correlation parameters R² and adjusted-R² for the model are 0.8275 and 0.7068, respectively.

View 1626 in Table 20 in FIG. 16K shows the parameter estimates, standard errors, t-values, and P-values. The pure quadratic term for relative humidity is the significant second order term. Although 0.176 is a high P-value for significance, one may consider the possibility of some curvature of the response surface. The fitted equation for inactivation of B. anthracis ΔSterne on InsulFab is:

y_{Ba(3)}=17.0315−2.0862b+3.0930x₁+3.5849x₂+3.7208x₃−1.6774x₁²−2.9997x₂²−1.6517x₃², (6)

where y_{Ba(3)} is the log odds of spore inactivation on the third InsulFab material. View 1345 in FIG. 13E shows a slice of the response surface at four days and blocking term b fixed at its mean value of 0.3333.

For B. thuringiensis Al Hakam on InsulFab, the ANOVA table for Sequential sum of squares in view 1628 at Table 21 in FIG. 16L shows that a first order model was needed. Neither two-way interactions nor pure quadratic effects were significant (p=0.9997 and p=0.9386, respectively). The F-test for lack of fit has a P-value of 0.340, which does not indicate lack of fit. Correlation parameters R² and adjusted-R² for the model are 0.7594 and 0.6906, respectively.

View 1630 at Table 22 at FIG. 16M shows the parameter estimates, standard errors, t-values, and P-values for B. thuringiensis Al Hakam on InsulFab. The corresponding fitted equation for inactivation is:

y_{Ba(3)}=12.0283+2.1611b+5.7042x₁+4.3161x₂+3.7813x₃, (7)
where Y_{Bt(3)} is the log odds of spore inactivation on the third material. View 1350 in FIG. 13F shows a slice of the response surface at four days and blocking term b fixed at its mean value of 0.3158.

On nylon, B. anthracis ΔSterne proved difficult to model as well as comparatively more difficult to inactivate. Using residual plots, three problematic test runs, one, three and fifteen, were identified. These observations were omitted and the model refitted with the caveat that the results for nylon are not as dependable as the results for other coupons. The ANOVA table for Sequential sum of squares in view 1632 at Table 23 in FIG. 16N shows a second order model was needed with pure quadratic terms and two-way interactions. The F-test for lack of fit has a P-value of 0.141, which does not indicate lack of fit. Correlation parameters R² and adjusted-R² for the model are 0.9972 and 0.9917, respectively.

View 1634 at Table 24 in FIG. 16O for B. anthracis ΔSterne on nylon shows the parameter estimates, standard errors, t-values, and P-values. The fitted equation for inactivation of B. anthracis ΔSterne on nylon is

y_{Ba(4)}=0.07582+2.1487b+6.0157x₁+4.6424x₂+1.3926x₃+4.2282x₁x₂−0.8748x₁x₃−0.3612x₂x₃+5.3117x₁²+0.6467x₂²−2.3881x₃², (8)

where Y_{Ba(4)} is the log odds of spore inactivation on the fourth nylon material. FIG. 13G shows a slice of the response surface at four days and blocking term b fixed at its mean value of 0.3125.

For B. thuringiensis Al Hakam on nylon, test runs one and fourteen were difficult to fit. These runs were omitted, and the model was refit. Interestingly, the number of days of treatment was not a significant predictor for this coupon p=0.664, so the duration was excluded from the model. The ANOVA table for Sequential sum of squares in view 1636 at Table 26 in FIG. 16P shows that a second order model with pure quadratic terms and two-way interactions was needed. The F-test for lack of fit has a P-value of 0.698, which does not indicate lack of fit. Correlation parameters R² and adjusted-R² for the model are 0.9778 and 0.9644, respectively.

View 1638 at Table 27 in FIG. 16Q shows the parameter estimates, standard errors, t-values, and P-values for B. thuringiensis Al Hakam on nylon. The corresponding fitted equation for inactivation is:

y_{Bt(4)}=−0.2778+0.1487b+4.5440x₁+4.0575x₂+0.0x₃+3.9837x₁x₂+0.0x₁x₃+0.0x₂x₃+1.5441x₁²+2.6875x₂²+0.0x₃² (9)

where Y_{Bt(4)} is the log odds of spore inactivation on the fourth material. View 1360 in FIG. 13H shows the response surface with blocking term b fixed at its mean value of 0.2941.

For B. anthracis ΔSterne on wiring, most observations had complete inactivation of spores and could not be modeled using RSM. All runs from view 1602 at Table 10 in FIG. 16A had complete inactivation except for runs two, three, nine, and twelve. The notable thing about these four points is that they all had two or more predictors at the low setting; i.e., at least two of x₁, x₂, or x₃ were equal to minus one for these four observations. This was considered as the “back corner” of the predictor space. The view 1410 in FIG. 14A highlights these points. Excepting these four points, the model would be y=1 or, equivalently, p=1. The results for B. anthracis ΔSterne on wiring also apply to B. thuringiensis Al Hakam.

For B. anthracis ΔSterne on polypropylene, run number one, a center run, was deemed an outlier and thus omitted for analysis. The corresponding ANOVA table for Sequential sum of squares in view 1642 at Table 28 in FIG. 16S shows that a second order model with pure quadratic terms should be considered. View 1642 at Table 28 in FIG. 16S shows that the significant pure quadratic term is relative humidity. Adding two-way interaction terms did not contribute-significantly to the model (p=0.641) and were not included. The F-test for lack of fit has a P-value of 0.187, which does not indicate lack of fit. Correlation parameters R²_ and adjusted-R² for the model are 0.8713 and 0.7811, respectively.

View 1642 at Table 28 in FIG. 16S shows the parameter estimates, standard errors, t-values, and P-values for B. anthracis ΔSterne on polypropylene. The corresponding fitted equation for inactivation of is:

$$\begin{matrix}{y}_{Ba ( 5 )} = 6.4025 + 6.4256 b + 6.9452 {x}_{1} + 2.2431 {x}_{2} + 4.364 {x}_{3} \\ = 1.0017 {x}_{1}^{2} + 4.8915 {x}_{2}^{2} - 2.3429 {x}_{3}^{2} ,\end{matrix} \qquad ( 10 )$$

where Y_{Ba(5)} is the log odds of spore inactivation on the fifth material polypropylene. View 1365 in FIG. 13I shows a slice of the response surface at four days and blocking term b fixed at its mean value of 0.3333.

Complete inactivation of B. thuringiensis Al Hakam spores on polypropylene was measured after test runs five, seven, fifteen, seventeen and nineteen. These observations are pictured in the design box view 1440 FIG. 14B. These data prevented the fitting of a second order model. Going beyond the usual RSM framework, however, a piecewise model was fitted using a second order model with pure quadratic terms for the points of incomplete activation and mapping the observations with complete inactivation to unity expressed in model as y=1 or, equivalently, p=1. The enclosed region 1455 in FIG. 14C has the log odds of inactivation mapped to infinity and the complement fitted as

y_{Bt(5)}=1.4271+0.7180b+1.7227x₁−0.1465x₂+0.3641x₃−0.0792x₁²+0.8467x₂²−0.5264x₃² (11)

where Y_{Bt(5)} is the log odds of spore inactivation for the fifth polypropylene material. The ANOVA table for Sequential sum of squares in view 1644 at Table 29 in FIG. 16T shows the significance of the pure quadratic terms for that part of the model without complete inactivation. View 1646 at Table 30 in FIG. 16U shows that relative humidity is the significant second order term. Two-way interactions were not significant (p=0.449). The F-test for lack of fit has a P-value of 0.452, which does not indicate lack of fit.

View 1646 at Table 30 at FIG. 16U shows the parameter estimates, standard errors, t-values, and P-values for B. thuringiensis Al Hakam spores on polypropylene. View 1370 in FIG. 13J shows a slice of the response surface at four days and blocking term b fixed at its mean value of 0.2308. For visual presentation, y=20 was used instead of y=1 to keep the scale reasonable but still show the piecewise behavior of the model.

For B. anthracis ΔSterne in wet solution, most observations for wet solution had complete inactivation of spores, and could not be modeled using RSM. All B. anthracis ΔSterne runs from view 1602 at Table 10 in FIG. 16A had complete inactivation except for runs three, six, and nine. The notable aspect about these three points is that they all had temperature at the low setting. FIG. 14D highlights these points 1465 where live spores were culturable.

For B. thuringiensis Al Hakam in wet solution, all test runs from view 1602 at Table 10 in FIG. 16A had complete inactivation except for runs one, three, six, nine, and twelve. Except for run one, a center point, these observations have temperature at the low setting. FIG. 14E highlights these points 1475 where live spores were culturable.

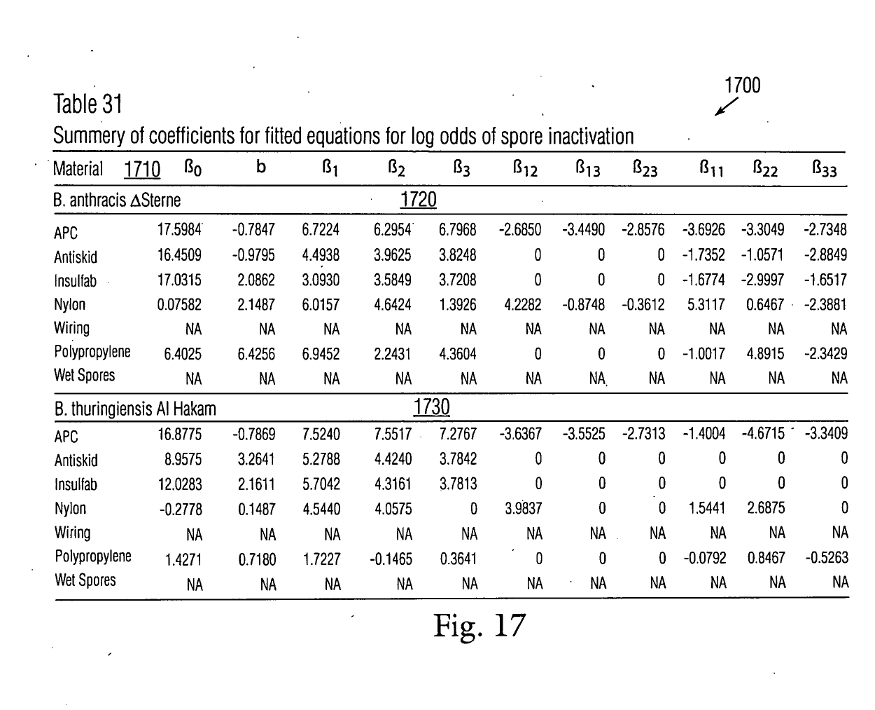

View 1700 at Table 32 in FIG. 17 provides a compact summary of all the fitted coefficients in the equations for log odds of spore inactivation. The row of coefficients 1710 include the boundary value β₀, the blocking term b, and factors β₁, β₂, β₃, β₁₂, β₁₃, β₂₃, β₁₁, β₂₂, and β₃₃ from eqn. (1). The values corresponding to the tested substrate material are provided in the first array 1720 for B. anthracis ΔSterne, and in the second array 1730 for B. thuringiensis Al Hakam.

Control driven test method improvements and the use of multiple independent spore preparations with a single protocol useful for both B. anthracis ΔSterne and B. thuringiensis Al Hakam enables the application of a statistically based experimental design, specifically RSM. This use of RSM permits subsequent mathematical analysis and modeling of the response, generating a predictive capability valuable to potential users of hot, humid air decontamination technology. These advancements in data analyses and interpretation, when coupled with decreased test time and cost illustrated in the literature, serve to better support the transition of decontamination technologies from the laboratory to the field.

In all modeled strain-material pairs but one, all three predictors (temperature, relative humidity, and time) were significant and each had an effect on spore inactivation. The exception was B. thuringiensis Al Hakam spores dried onto nylon webbing material, where time was not significant. Interestingly, for B. anthracis ΔSterne on two materials (InsulFab and polypropylene), relative humidity was the most significant second order pure quadratic term.

The most difficult materials to model were nylon webbing and polypropylene (dried plastic tubes) with a piecewise modeling strategy required for B. thuringiensis Al Hakam on polypropylene. Nylon was the only material for which multiple runs were omitted based on examination of residual plots. Hot, humid air was least effective at inactivating spores on nylon. This may be due to the porous, hydrophobic properties of nylon.

For wet spores and those dried onto wiring insulation, the majority of test runs showed complete inactivation. For wet spores of both strains, the cases without complete inactivation were at the lowest setting for temperature with only one exception. For spores of both strains on wiring insulation, the conditions without complete inactivation all had two or more predictors at the lowest settings.

This disclosure demonstrates the use of statistical methods to design biological decontamination experiments at the limits of decontamination technology and to analyze the resultant data. The fitted equations specific to strain and material permit a broad range of end-users with varying requirements and constraints the ability to predict the success of hot, humid air treatments on specific materials based on achievable temperature and relative humidity conditions and permissible decontamination times.

The objective of Tier II testing was to collect and analyze hot humid air sporicidal data over a performance range that included time, temperature and relative humidity for high purity Bacillus spores inoculated on different materials. Preliminary Tier I research published in NSWCDD/TR-11/161 indicated that hot humid air was an effective sporicide against four Bacillus strains in testing across three different military laboratories. Spores of virulent B. anthracis Ames, attenuated B. anthracis ΔSterne, and B. thuringiensis Al Hakam showed similar susceptibility to hot humid air in Tier I. Following Tier I, two strains were selected for further study during Tier II: B. anthracis ΔSterne and B. thuringiensis Al Hakam. A single spore preparation protocol was developed for both test strains that reproducibly yielded high purity spores.

Development of this protocol was critical to eliminate variables, demonstrate that both strains could be prepared under identical conditions, isolate spores as an independent variable, and to establish agent-simulant relationships. Furthermore, this preparation protocol for purified spores laid the foundation for future work to include statistical analysis of experimental data involving spore preparation variables such as debris and additives. Spores (>7 logs) of both strains were dried on six different test materials. Control-driven test methods were developed and combined with response surface methodology in order to identify the limits of spore survival at optimal test combinations of temperature (60° C., 68° C., 77° C.), relative humidity (60%, 75%, 90%) and time (1 days, 4 days, 7 days). No spores survived the harshest test run (77° C., 90% H_{R}, 7 days), while >6.5 logs of spores survived the mildest test run (60° C., 60% H_{R}, 1 day).

Spores of both strains inoculated on nylon webbing and polypropylene had greater survival rates at 68° C., 75% H_{R}, 4 days than spores on other materials. Electron microscopy showed no obvious physical damage to spores using hot, humid air, which contrasted with pH-adjusted bleach decontamination. Predictive models were then developed in order to evaluate the performance range of hot humid air decontamination. The models are described in this summary. Importantly, the impact of debris and/or additives on hot humid air decontamination kinetics remains unknown, and is expected to be evaluated with additional testing.

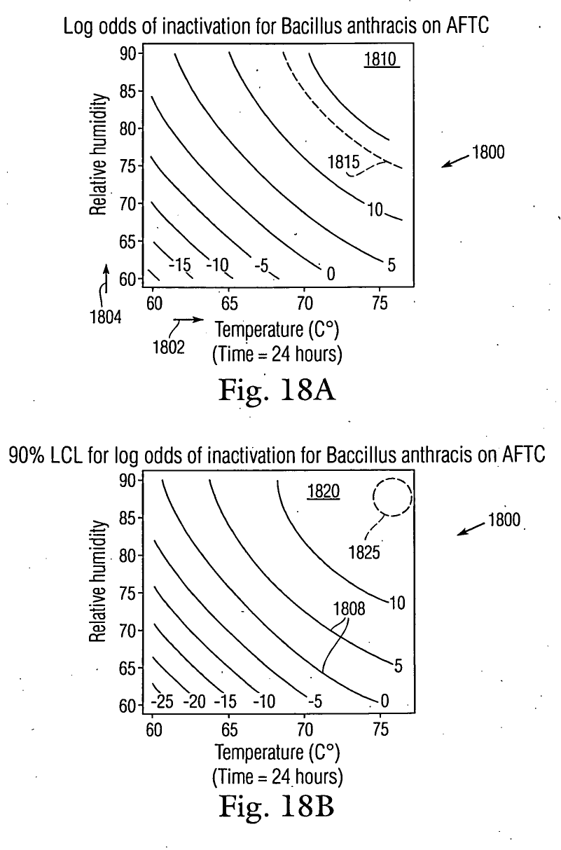

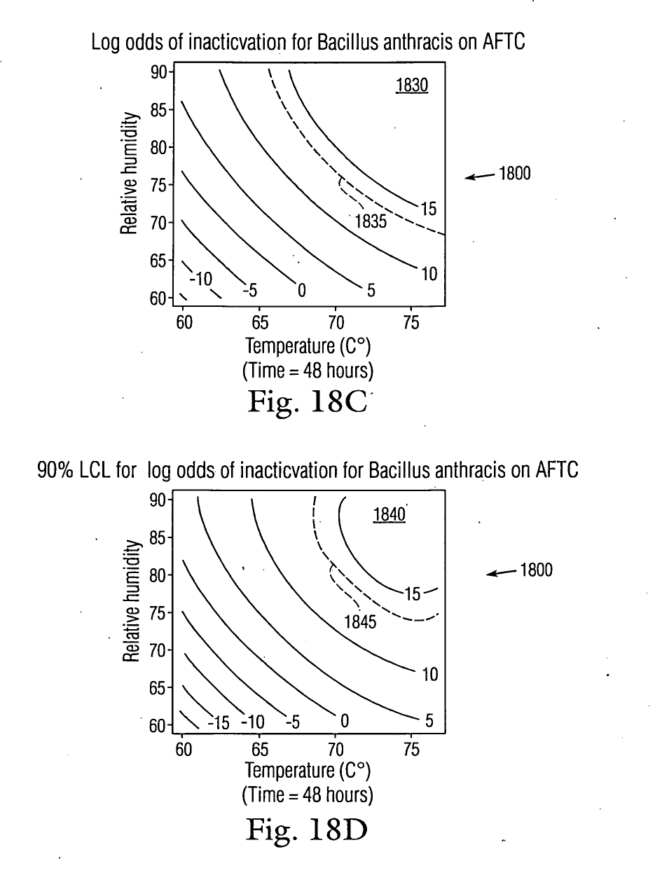

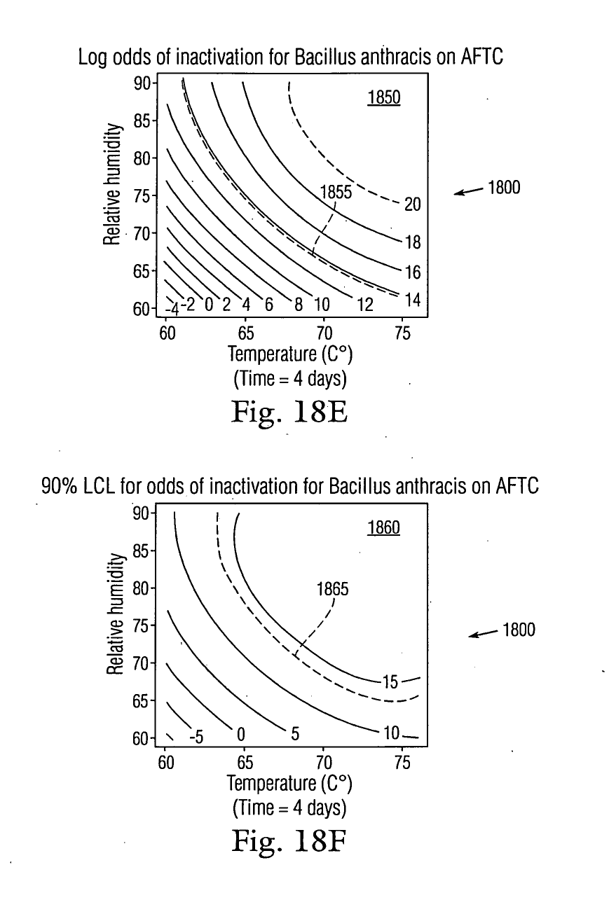

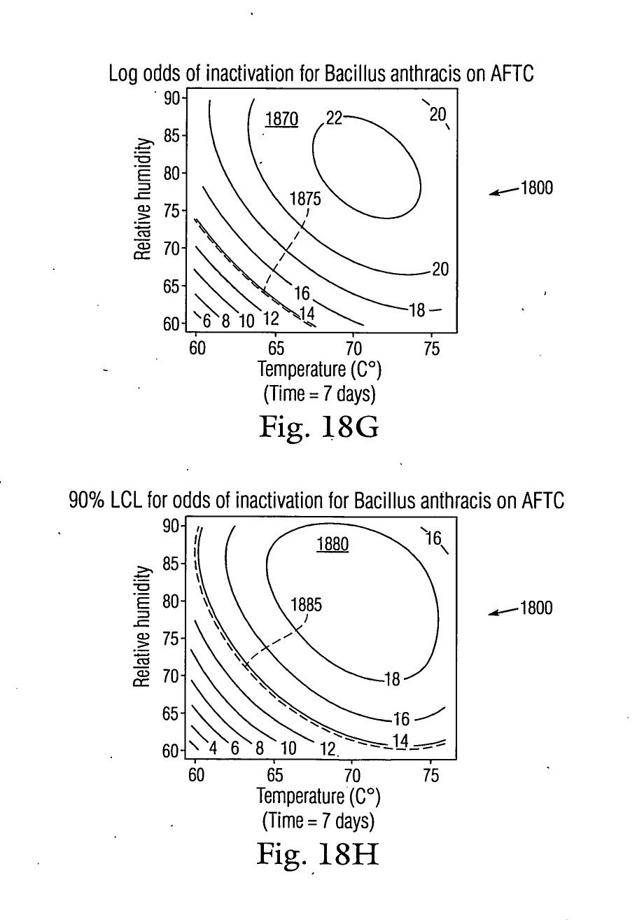

Logs odds values were fitted to predictive equations using response surface modeling (RSM) techniques. Two-dimensional contour plots were generated for the average values and the lower 90% confidence limit for fixed time points. Exemplary two-dimensional contour plots for spore inactivation on APC water dispersible aircraft paint on. aluminum 2024-T3, and nylon webbing are shown in FIGS. 18A through 21B.

All three predictors (temperature, relative humidity, and time) were significant and each had an effect on spore inactivation except for B. thuringiensis Al Hakam spores dried onto nylon, where time was not significant. Therefore, only a single two-dimensional contour plot for the experimental space of 1 to 7 days is shown for B. thuringiensis Al Hakam on nylon. Complete spore inactivation (>7 logs of spores) was achieved for wet spores and spores dried onto hydrophilic wiring insulation at all but the lowest hot humid air challenge of 140° F., 60% H_{R}, 1 day. Therefore these data were not modeled using RSM.

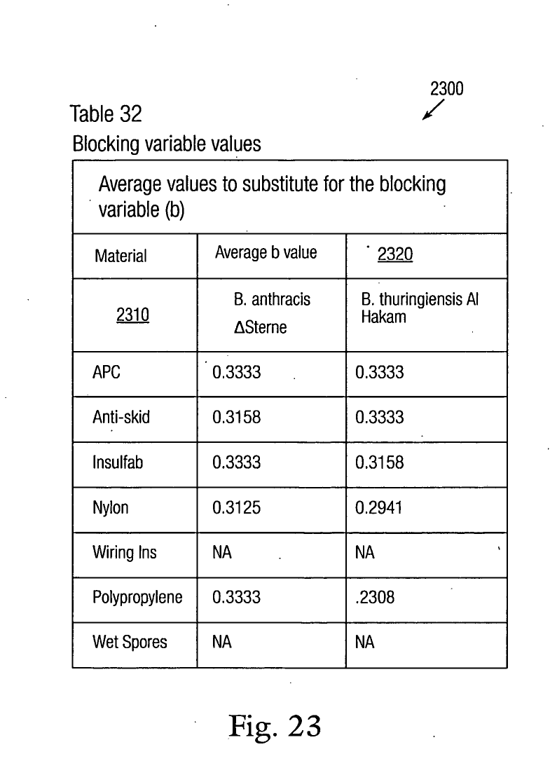

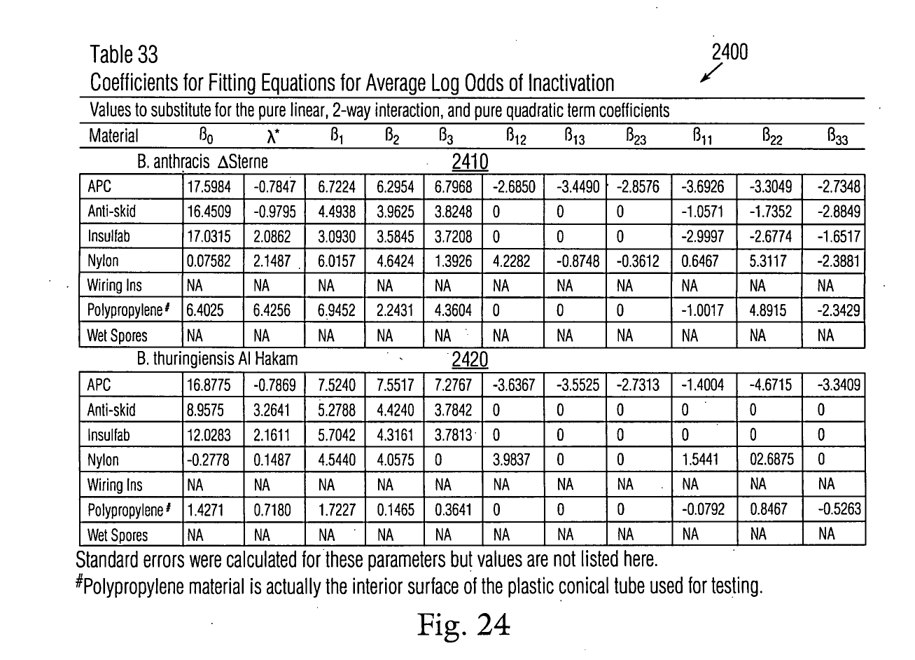

The fitted equations for the average log odds of inactivation are listed below for the strain/substrate pairs shown in FIGS. 18A-21B. All fitted equations for the average log odds of inactivation remaining strain/substrate pairs can be determined by using the generic equation presented later in this summary in conjunction with FIG. 23 in Table 32 and FIG. 24 in Table 33. For use in RSM analyses, the blocking variable b is used to indicate from which phase of the experiment an observation came. In the left hand side of the equations below, x is the vector (x₁, x₂, x₃). The expected average spore survival is calculated by solving the fitted equation for 1−p(x) and multiplying that value by the total number of spores tested (e.g., 7-logs).

FIGS. 18A through 18H present two-dimensional (2D) contour plot views 1800 of B. anthracis ΔSterne on APC (also referred to as AFTC). The abscissa 1802 denotes temperature and the ordinate 1804 denotes relative humidity. Solid contour curves 1806 (on the left-side plots) identify average log odds of inactivation, and similar contour curves 1808 (on the right-side plots) identify 90% lower confidence limit (LCL) constant values for those averages. FIG. 18A shows the log odds plot 1810 at 24 hours (one day) of treatment with hot humid air, and the dash contour curve 1815 represents the inactivation odds boundary of 6-logs (10⁶) of eqn. (1), with the shaded region above and to the right being greater than the boundary value. FIG. 18B shows the 90% LCL for log odds plot 1820, and the dash contour curve 1825 represents the inactivation odds boundary of 6-logs, with the shaded region being within the boundary of the dash curve 1825. These 6-log boundaries are displayed as visual aids to signify the effect of hot humid conditions on the spores.

FIGS. 18C and 18D illustrate 2D contour plot views for B. anthracis ΔSterne on APC at 48 hours (two days) of treatment with hot humid air. FIG. 18C shows the average log odds plot 1830 including the dash contour curve 1835 represents the inactivation odds boundary of 6-logs, with the shaded region at the upper right being greater than the boundary value. FIG. 18D shows the 90% LCL plot 1840 with the dash contour curve 1845 boundary and the shaded region at the upper right exceeding the LCL boundary value.

FIGS. 18E and 18F illustrate 2D contour plot views for B. anthracis ΔSterne on APC at four days of treatment with hot humid air. FIG. 18E shows the average log odds plot 1850 including the dash contour curve 1855 represents the inactivation odds boundary of 6-logs, with the shaded region at the upper right being greater than the boundary value. FIG. 18F shows the 90% LCL plot 1860 with the dash contour curve 1865 boundary and the shaded region at the upper right exceeding the LCL boundary value.

FIGS. 18G and 18H illustrate 2D contour plot views for B. anthracis ΔSterne on APC at seven days of treatment with hot humid air. FIG. 18G shows the average log odds plot 1880 including the dash contour curve 1885 represents the inactivation odds boundary of 6-logs, with the shaded region at the upper right being greater than the boundary value. FIG. 18H shows the 90% LCL plot 1890 with the dash contour curve 1895 boundary and the shaded region at the upper right exceeding the LCL boundary value.

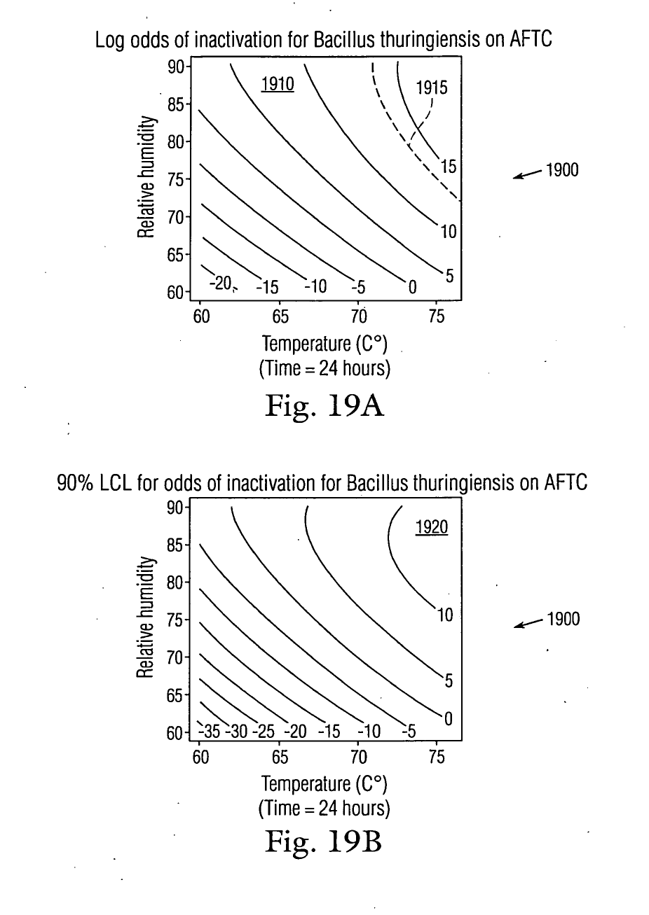

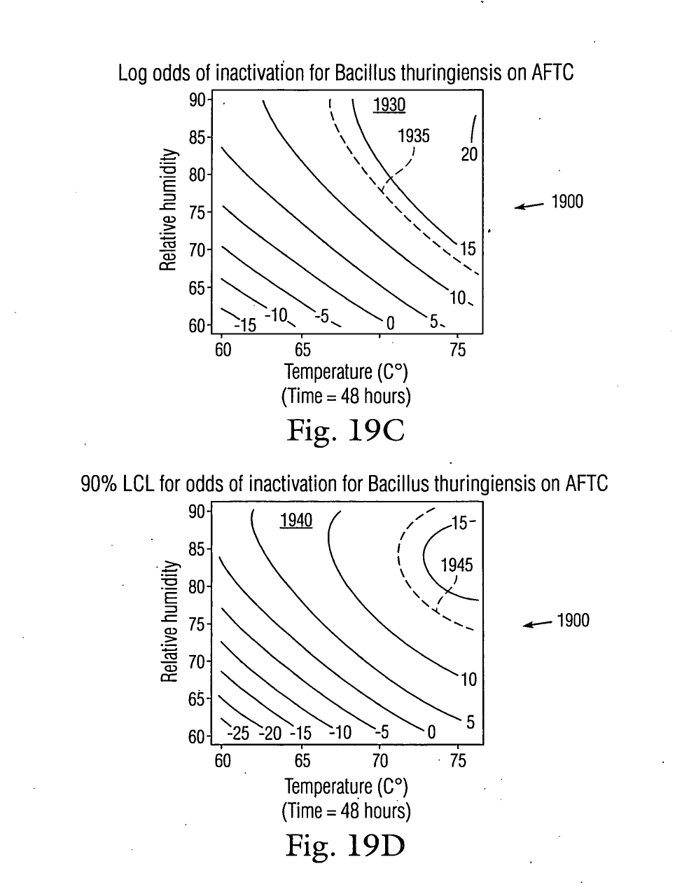

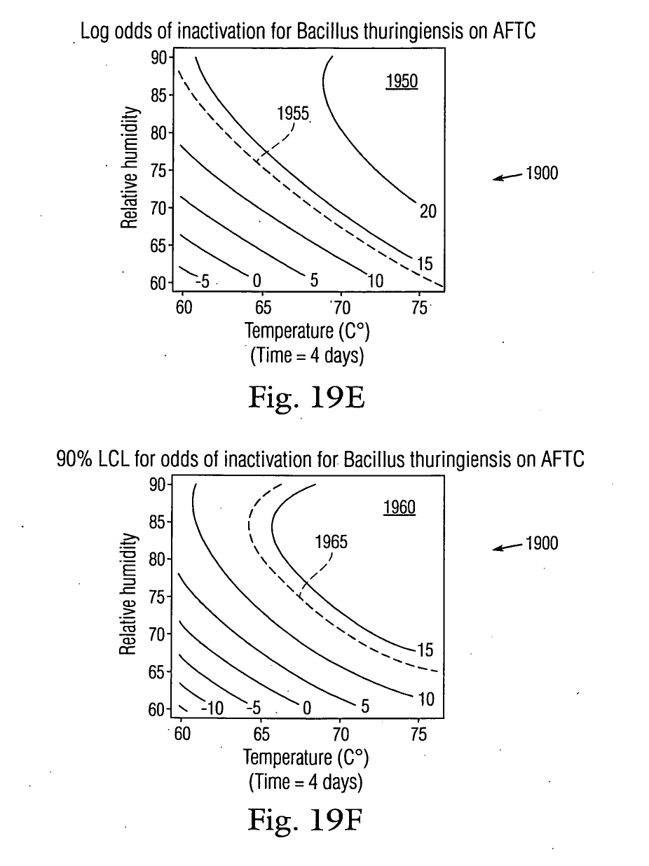

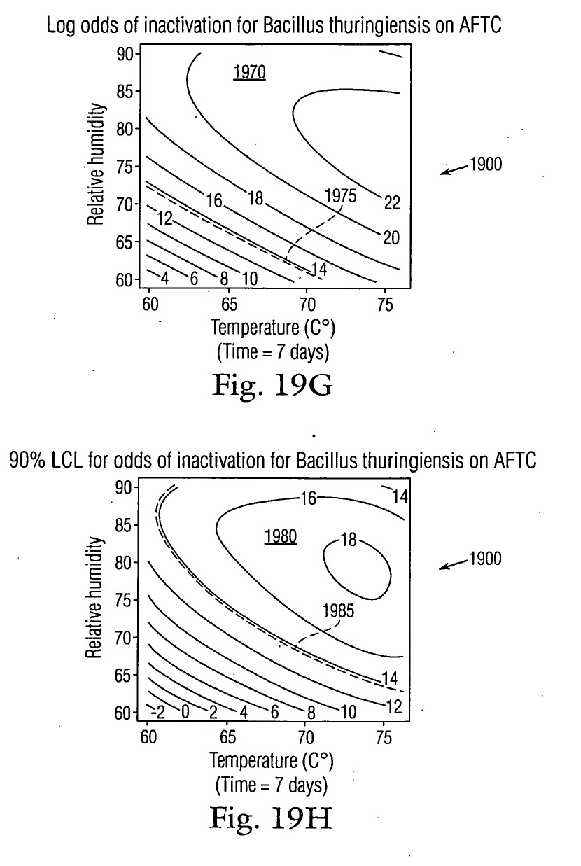

FIGS. 19A through 19H present 2D contour plot views 1900 of B. thuringinesis Al Hakam on APC. The temperature abscissa, relativity humidity ordinate and solid contour curves are comparatively similar to counterparts in views 1800. FIG. 19A shows the log odds plot 1910 at 24 hours (one day) of treatment with hot humid air, and the dash contour curve 1915 represents the inactivation odds boundary of 6-logs, with the shaded region in the upper right being greater than the one-day average boundary curve. FIG. 19B shows the 90% LCL for log odds plot 1920, and the dash contour curve 1925 represents the inactivation odds boundary of 6-logs, with the shaded region being within upper right region of that one-day LCL boundary curve.

FIG. 19C shows the log odds plot 1930 at two days of treatment with hot humid air, and the dash contour curve 1935 represents the inactivation odds boundary of 6-logs, with the shaded region in the upper right being greater than the boundary value. FIG. 19D shows the 90% LCL for log odds plot 1940, and the dash contour curve 1945 represents the inactivation odds boundary of 6-logs, with the shaded region being within upper right region of the dash curve 1945. FIG. 19E shows the log odds plot 1950 at four days of treatment with hot humid air, and the dash contour curve 1955 represents the inactivation odds boundary of 6-logs, with the shaded region in the upper right being greater than the two-day average boundary curve.

FIG. 19F shows the 90% LCL for log odds plot 1960, and the dash contour curve 1965 represents the inactivation odds boundary of 6-logs, with the shaded region being within upper right region of the four-day LCL boundary curve. FIG. 19G shows the log odds plot 1950 at seven days of treatment with hot humid air, and the dash contour curve 1955 represents the inactivation odds boundary of 6-logs, with the shaded region in the upper right being greater than the seven-day average boundary curve. FIG. 19H shows the 90% LCL for log odds plot 1980, and the dash contour curve 1985 represents the inactivation odds boundary of 6-logs, with the shaded region being within upper right region of that seven-day LCL boundary curve.

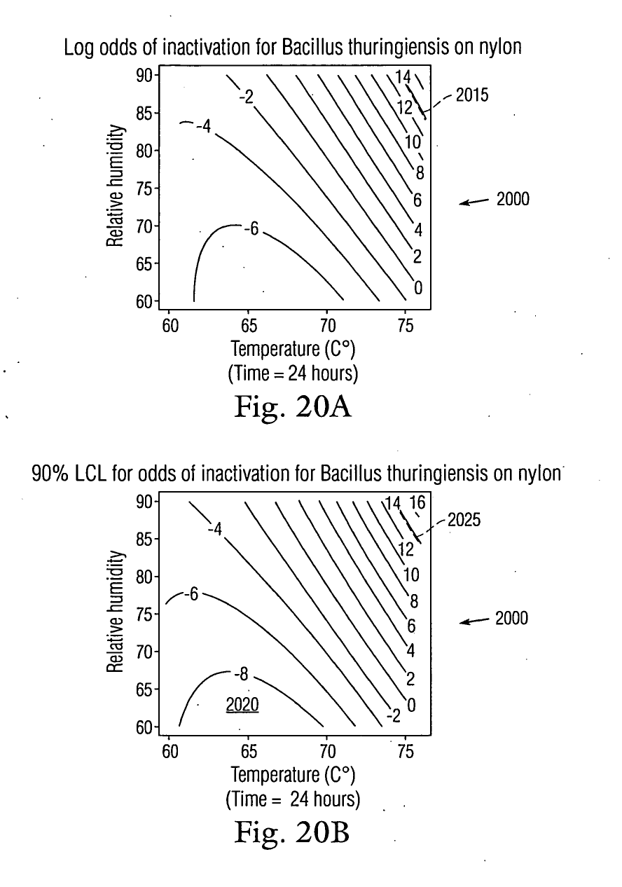

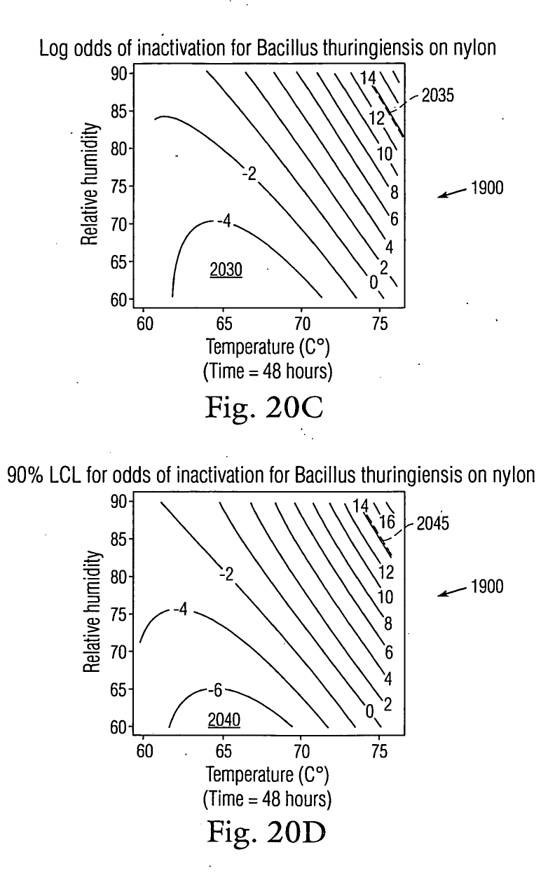

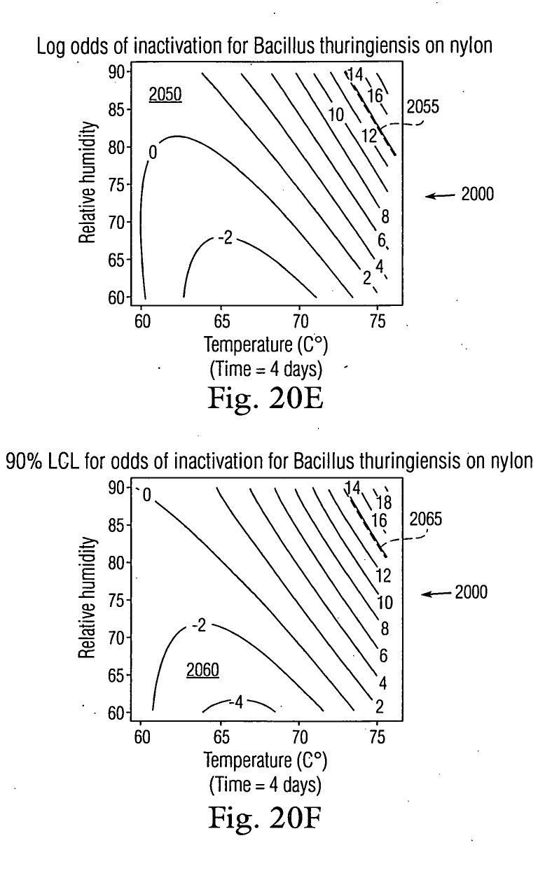

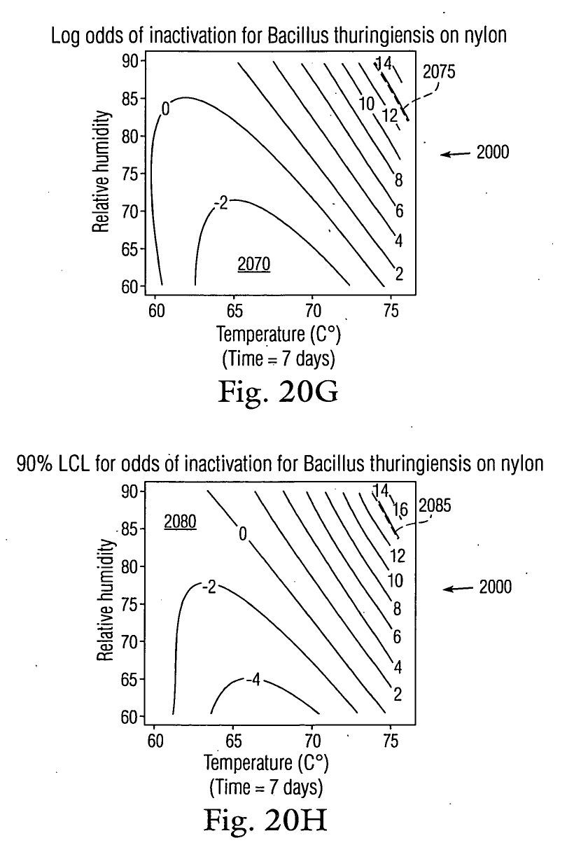

FIGS. 20A through 20H present 2D contour plot views 2000 of B. anthracis ΔSterne on nylon webbing. The temperature abscissa, relativity humidity ordinate and solid contour curves are comparatively similar to counterparts in views 1800. FIG. 20A shows the log odds plot 2010 at 24 hours (one day) of treatment with hot humid air, and the dash contour curve 2015 represents the inactivation odds boundary of 6-logs, with the shaded region in the upper right being greater than the one-day average boundary curve. FIG. 20B shows the 90% LCL for log odds plot 2020, and the dash contour curve 2025 represents the inactivation odds boundary of 6-logs, with the shaded region being within upper right region of that one-day LCL boundary curve.

FIG. 20C shows the log odds plot 2030 at two days of treatment with hot humid air, and the dash contour curve 2035 represents the inactivation odds boundary of 6-logs, with the shaded region in the upper right being greater than the boundary value. FIG. 20D shows the 90% LCL for log odds plot 2040, and the dash contour curve 2045 represents the inactivation odds boundary of 6-logs, with the shaded region being within upper right region of the dash curve 2045. FIG. 20E shows the log odds plot 2050 at four days of treatment with hot humid air, and the dash contour curve 2055 represents the inactivation odds boundary of 6-logs, with the shaded region in the upper right being greater than the two-day average boundary curve.

FIG. 20F shows the 90% LCL for log odds plot 2060, and the dash contour curve 2065 represents the inactivation odds boundary of 6-logs, with the shaded region being within upper right region of the four-day LCL boundary curve. FIG. 20G shows the log odds plot 2050 at seven days of treatment with hot humid air, and the dash contour curve 2055 represents the inactivation odds boundary of 6-logs, with the shaded region in the upper right being greater than the seven-day average boundary curve. FIG. 20H shows the 90% LCL for log odds plot 2080, and the dash contour curve 2085 represents the inactivation odds boundary of 6-logs, with the shaded region being within upper right region of that seven-day LCL boundary curve.

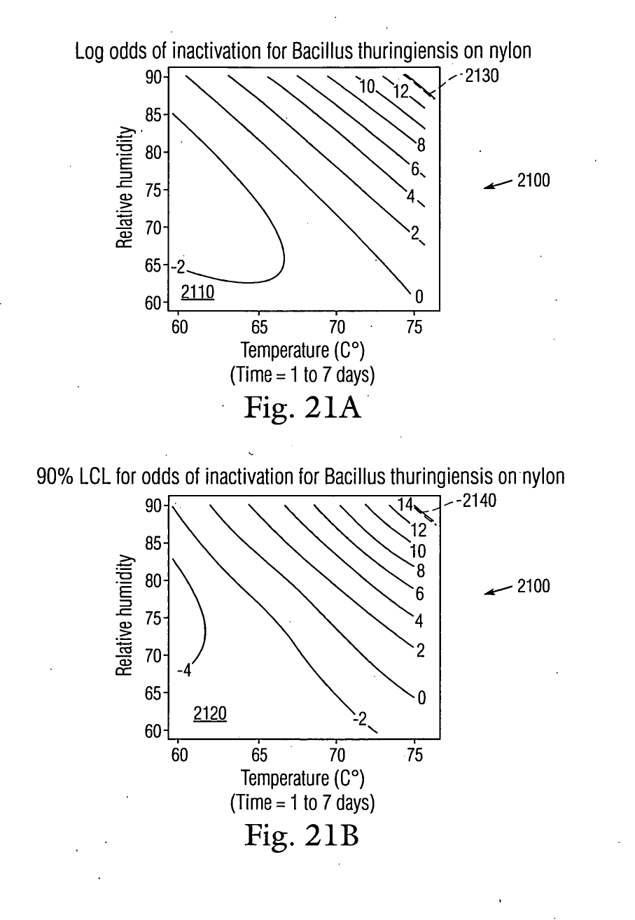

FIGS. 21A and 21B present 2D contour plot views 2100 of B. thuringinesis Al Hakam on nylon webbing between 1 day and seven days of treatment. The temperature abscissa, relativity humidity ordinate and solid contour curves are comparatively similar to counterparts in views 1800. FIG. 21A shows the log odds plot 2110 treated with hot humid air, and the dash contour curve 2115 represents the inactivation odds boundary of 6-logs, with the shaded region in the upper right being greater than the average boundary curve. FIG. 21B shows the 90% LCL for log odds plot 2120, and the dash contour curve 2125 represents the inactivation odds boundary of 6-logs, with the shaded region being within upper right region of that LCL boundary curve. Time was not a statistically significant predictor for this strain/material pair, so multiple plots with fixed time points were deemed unnecessary.

Single Spore Preparation Protocol is exemplified by the single spore preparation protocol in U.S. patent application Ser. No. 13/136,900 assigned Navy Case 100956 that was developed to prepare high purity spores of both B. thuringiensis Al Hakam and B. anthracis A Sterne. This protocol consistently yielded >95% phase-bright, heat-resistant, consistently sized spores at greater than 10⁸ spores mL⁻¹ of sporulation medium for both strains. Quality assurance was maintained though detailed datasheets that included spore titers during and after spore preparation, microscopy of spores, and spore heat resistance (65° C. for 30 minutes). Coulter analysis quantified the number of spores, determined spore size, and provided objective information on spore cleanliness.

The single preparation protocol was a necessary step forward to establish B. thuringiensis Al Hakam as a lead simulant for B. anthracis, due to B. thuringiensis Al Hakam spores being more difficult to prepare than others, as they tended to clump, presumably due to their hydrophobicity. All spores produced in the Tier II effort are high-purity, laboratory-grade benchmark spores. Eventually, other grades of spores should be tested (e.g., preparations containing dirt, organic debris, or powdering additives) as the presence of debris or additives may affect decontamination kinetics.

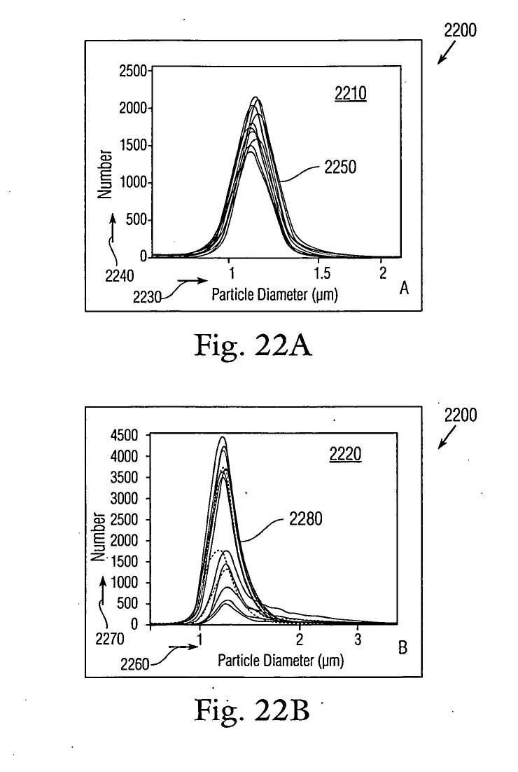

FIGS. 22A and 22B show views 2200 of NSWCDD Coulter profiles of spore preparations for (A) B. anthracis ΔSterne as plot 2210 and (B) B. thuringiensis Al Hakam as plot 2220. FIG. 22A shows the plot 2210 with abscissa 2230 as spore particle diameter in μm and ordinate 2240 as number of spores, with curves 2250 of sample tests reaching a concentration of about 1.2 μm and a peak number of spores of about two-thousand. FIG. 22B shows the plot 2220 with abscissa 2260 as spore particle diameter in μm and ordinate 2270 as number of spores, with curves 2280 of sample tests reaching a concentration of about 1.3 μm diameter with a peak of more than about four-thousand spores.

The exemplary Hot Humid Air Decontamination Biological Test Design was achieved by environmental test chambers used to control temperature and relative humidity (H_{R}) during testing. Test coupons (2×2 cm) inoculated with 7.1 to 7.8 logs of spores were contained inside 50 mL polypropylene conical tubes capped with 0.2-μm filter-caps 140. These tubes permitted air exchange while preventing spores from escaping the conical tube and NIST-certified hygrometer/thermometers 170 showed that all temperature and H_{R} targets were reached inside the filtered conical tubes within 40 minutes. Use of these tubes enabled pursuit of a test method with reduced handling of inoculated substrates. Also, a single-step extraction protocol with improved extraction efficiency useful for both strains on all substrates was developed. In the final protocol, spores were incubated in their original conical tubes for 60 minutes in an optimized nutrient-rich medium (1% glucose, 3% tryptic soy broth, 0.25% buffered peptone water, 0.05% Tween 80 at pH 7) prior to extraction via a multi-tube vortexer and plating on tryptic soy agar.

Also, the post-test conical tubes containing remaining extraction medium, substrates, and spores were incubated to qualitatively assess the viability of all remaining spores, including those not removed from the substrate. This method also introduced the use of wet spore controls. These control samples contained spores that were never dried, and served as the 100% recovery reference values. These data points were used to correct for extraction efficiency from different substrates. Five independent spore preparations for each strain were tested on six different materials: aircraft performance coating (APC), anti-skid, InsulFab, wiring insulation, nylon webbing, and the inside polypropylene surface of the conical test tubes.

No spores survived the harshest test run (77° C., 90% H_{R}, 7 days), while >6.5 logs of spores survived the mildest test run (60° C., 60% H_{R}, 1 day). Spores of both strains inoculated on nylon webbing and polypropylene (hydrophobic) had greater survival rates at 68° C., 75% H_{R}, 4 days compared to spores inoculated on other materials, particularly the hydrophilic substrate wiring insulation.

The exemplary Hot Humid Air Decontamination Design of Exerpiments (DOE)—with Response Surface Modeling involves the experimental design for Tier II being guided by statistical design of experiments (DOE), specifically response surface methodology (RSM). The use of RSM permitted the modeling of the response data and therefore generated a predictive capability for each substrate tested. RSM is used to model a process, often with a second order polynomial, in order to understand the process and maximize or minimize the response for certain setting of predictors (Beauregard et al. 1992, Montgomery 2009). The three factors evaluated as predictors were temperature (° C.), relative humidity (% H_{R}), and time (days). A three factor face-centered cube test design with two randomized blocks (Phase I and Phase II experiments) was used for this work.

Under multiple conditions, there was no spore survival. Because the response measured was the average number of surviving spores, the function logit(x) trends toward infinity in cases of no survival, thereby becoming difficult to model mathematically. To alleviate this problem in a way that conservatively estimates the probability of spore inactivation, 0.1+(number of spores tested) was substituted for zero in conditions with no spore survival.

Significant effects were determined by ANOVA, and first and second order models were either rejected or accepted using the F-test for lack of fit. For observed outliers or overly influential data points, the corresponding run was omitted from further RSM analysis (Montgomery 2009, Myers et al. 2009, Lenth 2009, Hosmer & Lemeshow 2000, Agresti 1996, Agresti 2002). The responses for substrate-spore strain pairs were ultimately modeled using eqn. (1), which includes a blocking term b with coefficient λ and coded variables for the predictors. The indicator variable b was used as a blocking parameter to account for any shift in the mean response that may have occurred between Phases I and II of the experiment. This blocking term is set to b=0 for observations taken in Phase I of the experiment. The blocking term is set to b=1 for observations taken in Phase II of the experiment.

The arithmetic average of block is usually used for the value of b in practical applications of the equation. These values are provided in view 2300 at Table 32 Average values to Substrate for the blocking variable in FIG. 23. The column 2310 lists substrate material, and the row 2320 identifies the Bacillus strain corresponding to the average blocking term. Alternatively, for blocking coefficient λ>0, b=0 provides a conservative estimate of inactivation and b=1 provides a more optimistic estimate. The reverse is true for strain-substrate pairs where λ<0.

The coefficients for all strain-substrate pairs are shown in view 2400 at Table 33 Coefficients for Fitted Equations for Average Log Odds of Inactivation in FIG. 24, similar to view 1700 at Table 31, but also including the coefficient λ in the listing. The first array 2410 provides the values for eqn. (1) corresponding to B. anthracia ΔSterne for the listed substrate materials. The second array 2420 provides the corresponding values for B. thuringiensis Al Hakam for these materials.

In all modeled strain-substrate pairs but one, all three predictors (temperature, relative humidity, and time) were significant, and each had an effect on spore inactivation. The exception was B. thuringiensis Al Hakam spores dried onto nylon webbing material, where time was not significant. Interestingly, for B. anthracis ΔSterne on two materials (InsulFab and polypropylene), relative humidity was the most significant second order pure quadratic term. In line with the hot humid air decontamination data, the most difficult materials to model were nylon webbing and polypropylene (dried plastic tubes); with a piecewise modeling strategy required for B. thuringiensis Al Hakam on polypropylene. Additionally, nylon was the only material for which multiple runs were omitted based on observation of residual plots.

For wet spores and those dried onto wiring insulation, the majority of test runs showed complete inactivation and therefore these data sets could not be modeled (coefficients in table are listed as NA, or not applicable). Complete inactivation of wet spores occurred under all conditions except the lowest setting for temperature, with only one exception. For spores of both strains on wiring insulation, all conditions without complete inactivation had two or more predictors at the lowest settings.

These efforts present the current developments for modeling of pathogen neutralization. The comparative quantification using contour maps enables decontamination procedures to be employed with greater confidence of expectant success.

While certain features of the embodiments of the invention have been illustrated as described herein, many modifications, substitutions, changes and equivalents will now occur to those skilled in the art. It is, therefore, to be understood that the appended claims are intended to cover all such modifications and changes as fall within the true spirit of the embodiments.

What is claimed is:

1. A protocol method for analyzing survival of Bacillus spores on a substrate, said method comprising:
isolating a sample of the spores;
providing an environmental matrix of exposure condition elements, said matrix having control ranges of temperature, relative humidity and exposure duration, said control ranges extending from a low extremity to a high extremity;
determining a midpoint for each control range between said low and high extremities;
selecting condition elements from said matrix, with each condition element corresponding to a first condition being at one of said low and high range extremities and remaining conditions occupying said midpoint for a corresponding control range, wherein all condition elements are uniquely distinguishable;
subjecting said sample to said condition elements from said matrix;
counting survival spores for each said condition element;
performing a response surface methodology (RSM) analysis from said survival spores; and
determining coefficients that satisfy a relation for k conditions:
$$ln ( \frac{p ( x )}{1 - p ( x )} ) = {β}_{0} + {\sum }_{j = 1}^{k} {β}_{j} {x}_{j} + {\sum }_{j = 1}^{k} {β}_{jj} {x}_{j}^{2} + {\sum }_{i < j}^{ } {\sum }_{j = 2}^{k} {β}_{ij} {x}_{i} {x}_{j} + b .$$
where proportion response p(x) of inactivated spores that quantifies long odds spore inactivation, xⱼ represents a set of independent variables, βⱼ denotes a correlation coefficient from j=0, 1, . . . k, and b represents a correlation blocking term.

2. The method according to claim 1, wherein the spores are one of B. anthracis ΔSterne and B. thuringiensis Al Hakam.
